(19) United States
(12) Patent Application Publication
(10) Pub. No.: US 2021/0280275 A1
(43) Pub. Date: Sep. 9, 2021

(54) SYSTEMS AND METHODS FOR ANALYSIS OF ALTERNATIVE SPLICING

(71) Applicant: Envisagenics, Inc., New York, NY (US)

(72) Inventors: Martin AKERMAN, New York, NY (US); Maria Luisa PINEDA, New York, NY (US)

(21) Appl. No.: 16/952,231

(22) Filed: Nov. 19, 2020

Related U.S. Application Data
(63) Continuation of application No. PCT/US2019/033574, filed on May 22, 2019.
(60) Provisional application No. 62/675,590, filed on May 23, 2018.

(51) Int. Cl. G16B 50/30; G16B 5/20; G16B 25/10; G16B 40/30; G16H 20/10; G16H 20/30; G16H 50/20; G16H 50/30

(52) U.S. Cl. CPC G16B 50/30; G16B 5/20; G16B 25/10; G16H 50/30; G16H 20/10; G16H 20/30; G16H 50/20; G16B 40/30

(57) ABSTRACT

Disclosed herein are systems and methods for quantification and analysis of alternative splicing events, and prediction of biological relevance of alternative splicing events comprising a software module: quantifying alternative splicing events using biological data related to a genome, a transcriptome, or both provided by a user; processing the quantified alternative splicing events with information stored in a database; identifying statistically significant alternative splicing events, predicting functional impact of alternative splicing events on protein structures, protein functions, RNA stability, RNA integrity, or biological pathways, predicting druggability and reversibility of aberrant splicing events as well as controllability of splicing in general using statistical modeling and machine learning algorithms

### CROSS-REFERENCE

This application is a Continuation of International Patent Application No. PCT/US2019/033574, filed on May 22, 2019, which claims the benefit of U.S. Provisional Patent Application No. 62/675,590, filed on May 23, 2018, each of which is hereby incorporated by reference in its entirety for all purposes.

### STATEMENT AS TO FEDERALLY SPONSORED RESEARCH

This invention was made with U.S. government support, Grant Nos. 1R43GM116478-01 and 2R44GM116478-02A1, awarded by National Institute of Health under the Department of Health and Human Services. The U.S. government has certain rights to the invention.

### BACKGROUND

Cancer and genetic diseases affect more than 30 million people in the U.S. Diseases like Myelodysplastic Syndrome, Acute Myeloid Leukemia, Amyotrophic Lateral Sclerosis, Huntington disease and Spinal Muscular Atrophy can be caused by errors in RNA Splicing. RNA splicing is the process by which introns, the non-protein coding regions of DNA, are removed from nascent precursor messenger RNA (pre-mRNA), and exons, the protein coding regions of DNA, are joined together to form mature messenger RNA (mRNA). RNA splicing errors result in spliced RNA that do not produce functional proteins, thereby causing genetic diseases including many types of cancers. The global RNA therapeutics market is predicted to be about $1.2B by 2020.

### SUMMARY

RNA splicing can deliver significant therapeutic potential. It has been reported that 370 genetic disorders are caused by splicing errors. Additionally, about 15% of all disease—causing mutations are predicted to disrupt splicing and about 50% of synonymous cancer-driver mutations impair splicing. Thus, there is an urgent and unmet need to discover aberrant splicing(s) that can be drug-targets and/or biomarkers, to accelerate drug innovation for a wide spectrum of diseases.

In one aspect, disclosed herein is a computer-implemented system for quantifying alternative splicing (AS) events comprising: a digital processing device comprising: a processor, an operating system configured to perform executable instructions, a memory, and a computer program including instructions executable by the digital processing device to create an alternative splicing quantification application, the alternative splicing quantification application comprising a software module for: receiving information from a user, the information comprising biological data related to a genome, a transcriptome, or both; mapping the information to a database to create mapped information; computing a set of data-dependent parameters from the mapped information using heuristic approximations; and applying a probability model to the set of data-dependent parameters to generate alternative splicing values. In some embodiments, the probability model is a Bayesian probability model. In some embodiments, the computing a set of data-dependent parameters from the mapped information is automatic. In some embodiments, the applying a probability model to the set of data-dependent parameters to generate alternative splicing values is automatic. In some embodiments, the computing a set of data-dependent parameters from the mapped information is executed only once for each DNA, RNA, or mRNA sequence of the biological data related to the genome. In some embodiments, the computing a set of data-dependent parameters from the mapped information is executed once for each DNA, RNA, or mRNA sequence of the biological data related to the genome. In some embodiments, the applying a probability model to the set of data-dependent parameters to generate alternative splicing values is executed only once for each DNA, RNA, or mRNA sequence of the biological data related to the genome. In some embodiments, the computing a set of data-dependent parameters from the mapped information is not adjusted by the user. In some embodiments, the applying a probability model to the set of data-dependent parameters to generate alternative splicing values is not adjusted by the user. In some embodiments, the set of data-dependent parameters comprises a fragment size distribution. In some embodiments, the computing further comprises heuristic approximation, the heuristic approximation comprising replacing an inclusion ratio model with a data-driven model or a mathematical model of inclusion ratio. In some embodiments, the alternative splicing values comprises an exon inclusion ratio or a percent spliced index (PSI). In some embodiments, the alternative splicing values are at an exon level. In some embodiments, the biological data related to a genome, a transcriptome, or both comprises one or more of: a DNA sequence, an RNA sequence, a pre-mRNA sequence, and a mRNA sequence. In some embodiments, the receiving information from a user is via a computer network comprising a cloud network. In some embodiments, the software module further comprises a user interface allowing a user to sort alternative splicing values, filter alternative splicing values, select information stored in the database, merge alternative splicing values with the selected information stored in the database, view the one or more statistically significant alternative splicing events, select alternative splicing events for prediction of functional impact thereof, or a combination thereof. In some embodiments, the system herein further comprises a software module allowing the user to sort, filter, or rank the one or more statistically significant alternative splicing events based on user-selected criteria.

In another aspect, disclosed herein is a computer-implemented system for analyzing alternative splicing events comprising: a digital processing device comprising: a processor, an operating system configured to perform executable instructions, a memory, and a computer program including instructions executable by the digital processing device to create an alternative splicing analysis application, the alternative splicing analysis application comprising a software module for: receiving information from a user, the information comprising biological data related to a genome, a transcriptome, or both; and processing the information quantitatively to identify one or more statistically significant alternative splicing events, comprising: calculating one or more parameters of a regression model; and applying the regression model to the information using the one or more parameters to identify the one or more statistically significant alternative splicing events. In some embodiments, the regression model is a Thin Plate Spline-based regression model. In some embodiments, information comprising an exon inclusion ratio is calculated from the information comprising the biological data related to a genome, a transcriptome, or both. In some embodiments, the regression model comprises a Thin Plate Spline (TPS) model. In some embodiments, the system herein further comprises a software module processing the one or more statistically significant alternative splicing events with additional information stored in a database or a second database to quantify reproducibility of alternative splicing events in public datasets, descriptive analytics based on clinical metadata, functional impact thereof on protein structure, protein function, RNA stability, RNA integrity, or biological pathways, druggability and reversibility of aberrant splicing events and controllability of splicing regulation, comprising quantitatively estimating probabilities of the one or more statistically significant alternative splicing events of damaging the protein structures, protein functions, RNA stability, RNA integrity, or biological pathways using a plurality of features, wherein the features are generated using the additional information stored in the database, wherein the additional information comprises metadata obtained from annotations of a plurality of splicing types of alternative splicing based on public RNA-seq data, CLIP-seq data, mRNA annotations, GTEx data, TCGA data, clinical metadata, protein structure information, or genomic data, and applying a supervised or semi-supervised machine learning algorithm to predict the functional impact of the one or more significant alternative splicing events based on the estimated probabilities. The computer-implemented system of claim 21, further comprising a software module generating the annotations, wherein the annotation comprises information related to public RNA-seq data. In some embodiments, the plurality of splicing types comprises one or more of: alternative acceptors (AA), alternative donors (AD), cassette exons (CA), and intron retention (IR). In some embodiments, the annotations comprise one or more selected from: (i) read coverage of every splice junction detected from public data; (ii) frequency and sample types in which a splice site is detected; (iii) likelihood to observe a given alternative splicing variant across a plurality of public samples; (iv) prevalence of alternative splicing events in primary cancers and metastasis, correlation to age, gender and ethnicity, associated survival and relapse rates, and molecular and histological biomarkers; (v) location of alternative splicing events on human genes; (vi) prevalence of alternative splicing events in normal human organs or tissues; (vii) customized features and predictions; and (viii) splicing regulatory interactions (RBP-RNA). In some embodiments, the annotations comprise one or more new annotations generated using information received from the user. In some embodiments, the system herein further comprises a semi-supervised or supervised machine learning classifier to differentiate between functional splicing regulatory elements and cryptic splicing regulatory elements of one or more of the alternative splicing events thereby predicting controllability of splicing, druggability and reversibility of aberrant splicing events. In some embodiments, the predicting controllability of splicing, druggability and reversibility of aberrant splicing events is configured to be utilized for interpreting splicing events. In some embodiments, the biological data related to a genome, a transcriptome, or both comprises one or more of: a DNA sequence, an RNA sequence, a pre-mRNA sequence, and a mRNA sequence. In some embodiments, the receiving information from a user is via a computer network comprising a cloud network. In some embodiments, the software module further comprises a user interface allowing a user to sort alternative splicing values, filter alternative splicing values, select information stored in the database, merge alternative splicing values with the selected information stored in the database, view the one or more statistically significant alternative splicing events, select alternative splicing events for prediction of functional impact thereof, or a combination thereof. In some embodiments, the system herein further comprises a software module allowing the user to sort, filter, or rank the one or more statistically significant alternative splicing events based on user-selected criteria.

In yet another aspect, disclosed herein is a computer-implemented system for quantifying functional impact of alternative splicing events on protein structures, protein functions, RNA stability, RNA integrity, or biological pathways comprising: a digital processing device comprising: a processor, an operating system configured to perform executable instructions, a memory, and a computer program including instructions executable by the digital processing device to create an alternative splicing functional impact analysis application, the application comprising a software module for: generating a plurality of features based on information stored in a database, wherein the information comprises metadata obtained from annotations of a plurality of types of alternative splicing based on public RNA-seq data or other biological data; obtaining one or more alternative splicing events; quantitatively estimating probabilities of the one or more alternative splicing events of damaging the protein structures, protein functions, RNA stability, RNA integrity, or biological pathways based on the plurality of features; applying a supervised or semi-supervised machine learning algorithm to predict the functional impact of the one or more alternative splicing events based on the estimated probabilities; and generating a list of prioritized and biologically relevant alternative splicing events based on prediction of the functional impact of the one or more alternative splicing events. In some embodiments, the semi-supervised or supervised machine learning algorithm comprises: a random forest, Bayesian model, a regression model, a neural network, a classification tree, a regression trees, discriminant analysis, a k-nearest neighbors method, a naive Bayes classifier, support vector machines (SVM), a generative model, a low-density separation method, a graph-based method, a heuristic approach, or a combination thereof. In some embodiments, the machine learning algorithm is trained with a training set, each data point of the training set comprising a feature of the plurality of features, and a label, the label being positive, negative, or unlabeled. In some embodiments, the training set comprises of no less than 50 training data points. In some embodiments, the plurality of features comprises one or more categories of features selected from: RNA-based features, protein domain features, evolutionary features, mutability features, and splicing regulatory features. In some embodiments, the quantitatively estimating probabilities of the one or more alternative splicing events of damaging the protein structures, protein functions, RNA stability, RNA integrity, or biological pathways comprises quantitatively estimating damage caused by: removal of a functional protein domain by alternative splicing; nonsense-mediated decay (NMD) and translation frameshifting (FS) by alternative splicing; mutability of alternative splicing events; weighted closeness centrality of alternatively spliced proteins in a biological network; or a combination thereof. In some embodiments, the annotations comprise one or more selected from: (i) read coverage of every splice junction detected from public data; (ii) frequency and sample types in which a splice site is detected; (iii) likelihood to observe a given alternative splicing variant across a plurality of public samples; (iv) prevalence of alternative splicing events in primary cancers and metastasis, correlation to age, gender and ethnicity, associated survival and relapse rates, and molecular and histological biomarkers; (v) location of alternative splicing events on human genes; (vi) prevalence of alternative splicing events in normal human organs or tissues; (vii) customized features and predictions; and (viii) splicing regulatory interactions (RBP-RNA).

In yet another aspect, disclosed herein is a computer-implemented system for analyzing alternative splicing events comprising: a digital processing device comprising: a processor, an operating system configured to perform executable instructions, and a memory; a computer program including instructions executable by the digital processing device; a database configured to allow automatic interrogation of alternative splicing events through exon-centric data mapping, wherein each entry of the database comprises an independent alternative splicing event and wherein the database comprises one or more annotations generated using biological data related to a genome, a transcriptome, or both, the biological data provided by a user of the database; and a software module distributing analysis of a first plurality of alternative splicing events to a second plurality of processors. In some embodiments, the first plurality of splicing events is distributed via a computer network.

In still yet another aspect, disclosed herein is a computer-implemented method for quantifying alternative splicing (AS) events comprising: receiving information from a user, the information comprising biological data related to a genome, a transcriptome, or both; mapping the information to a database to create mapped information; computing a set of data-dependent parameters from the mapped information using heuristic approximations; and applying a probability model to the set of data-dependent parameters to generate alternative splicing values. In some embodiments, the biological data related to a genome, a transcriptome, or both comprises one or more of: a DNA sequence, a RNA sequence, a pre-mRNA sequence, or a mRNA sequence. In some embodiments, receiving information from a user is via a computer network comprising a cloud network.

In still yet another aspect, disclosed herein is a computer-implemented method for analyzing alternative splicing (AS) events comprising: receiving information from a user, the information comprising biological data related to a genome, a transcriptome, or both; and processing the information quantitatively to identify one or more statistically significant alternative splicing events, comprising: calculating one or more parameters of a regression model; and applying the regression model to the information using the one or more parameters to identify the one or more statistically significant alternative splicing events. In some embodiments, the probability model is a Bayesian probability model. In some embodiments, the regression model is a Thin Plate Spline-based regression model. In some embodiments, the biological data related to a genome, a transcriptome, or both comprises one or more of: a DNA sequence, a RNA sequence, a pre-mRNA sequence, or a mRNA sequence. In some embodiments, receiving information from a user is via a computer network comprising a cloud network. In some embodiments, the method herein further comprises allowing a user to sort alternative splicing values, filter alternative splicing values, select information stored in the database, merge alternative splicing values with the selected information stored in the database, view the one or more statistically significant alternative splicing events, select alternative splicing events for prediction of functional impact thereof, or a combination thereof. In some embodiments, an exon inclusion ratio is calculated from the information comprising the biological data related to a genome, a transcriptome, or both. In some embodiments, the regression model comprises a Thin Plate Spline (TPS) model. In some embodiments, the computing a set of data-dependent parameters from the mapped information is automatic. In some embodiments, the applying a probability model to the set of data-dependent parameters to generate alternative splicing values is automatic. In some embodiments, the computing a set of data-dependent parameters from the mapped information is executed only once for each DNA, RNA, or mRNA sequence of the biological data related to the genome. In some embodiments, the computing a set of data-dependent parameters from the mapped information is executed once for each DNA, RNA, or mRNA sequence of the biological data related to the genome. In some embodiments, the applying a probability model to generate alternative splicing values is executed only once for each DNA, RNA, or mRNA sequence of the biological data related to the genome. In some embodiments, the computing a set of data-dependent parameters from the mapped information is not adjusted by the user. In some embodiments, the applying a probability model to generate alternative splicing values is not adjusted by the user. In some embodiments, said one of the set of data-dependent parameters comprises a fragment size distribution. In some embodiments, the computing further comprises heuristic approximation, the heuristic approximation comprising replacing an inclusion ratio model with a data-driven model or a mathematical model of inclusion ratio. In some embodiments, the alternative splicing values comprises an exon inclusion ratio or a percent spliced index (PSI). In some embodiments, the alternative splicing values are at an exon level. In some embodiments, the method herein further comprises processing the one or more statistically significant alternative splicing events with additional information stored in a database or a second database to quantify reproducibility of alternative splicing events in public datasets, descriptive analytics based on clinical metadata, functional impact thereof on protein structure, protein function, RNA stability, RNA integrity, or biological pathways, druggability and reversibility of aberrant splicing events and controllability of splicing regulation, comprising quantitatively estimating probabilities of the one or more statistically significant alternative splicing events of damaging the protein structures, protein functions, RNA stability, RNA integrity, or biological pathways using a plurality of features, wherein the features are generated using the additional information stored in the database, wherein the additional information comprises metadata obtained from annotations of a plurality of splicing types of alternative splicing based on public RNA-seq data, CLIP-seq data, mRNA annotations, GTEx data, TCGA data, clinical metadata, protein structure information, or genomic data, and applying a supervised or semi-supervised machine learning algorithm to predict the functional impact of the one or more significant alternative splicing events based on the estimated probabilities. In some embodiments, the method herein further comprises generating the annotations, wherein the annotation comprises information related to public RNA-seq data. In some embodiments, the plurality of splicing types comprises one or more of: alternative acceptors (AA), alternative donors (AD), cassette exons (CA), and intron retention (IR). In some embodiments, the annotations comprise one or more selected from: (i) read coverage of every splice junction detected from public data; (ii) frequency and sample types in which a splice site is detected; (iii) likelihood to observe a given alternative splicing variant across a plurality of public samples; (iv) prevalence of alternative splicing events in primary cancers and metastasis, correlation to age, gender and ethnicity, associated survival and relapse rates, and molecular and histological biomarkers; (v) location of alternative splicing events on human genes; (vi) prevalence of alternative splicing events in normal human organs or tissues; (vii) customized features and predictions; and (viii) splicing regulatory interactions (RBP-RNA). In some embodiments, the annotations comprise one or more new annotations generated using information received from the user. In some embodiments, the method herein further comprises a semi-supervised or supervised machine learning classifier to differentiate between functional splicing regulatory elements and cryptic splicing regulatory elements of one or more of the alternative splicing events thereby predicting controllability of splicing, druggability and reversibility of aberrant splicing events. In some embodiments, the predicting controllability of splicing, druggability and reversibility of aberrant splicing events is configured to be utilized for interpreting splicing events. In some embodiments, the method herein further comprising a software module allowing the user to sort, filter, or rank the one or more statistically significant alternative splicing events based on user-selected criteria.

In yet another aspect, disclosed herein is a computer-implemented method for quantifying a functional impact of alternative splicing events on protein structures, protein functions, RNA stability, RNA integrity, or biological pathways comprising: generating a plurality of features based on information stored in a database, wherein the information comprises metadata obtained from annotations of a plurality of types of alternative splicing based on public RNA-seq data or other biological data; obtaining one or more alternative splicing events; quantitatively estimating probabilities of the one or more alternative splicing events of damaging the protein structures, protein functions, RNA stability, RNA integrity, or biological pathways based on the plurality of features; applying a supervised or semi-supervised machine learning algorithm to predict the functional impact of the one or more alternative splicing events based on the estimated probabilities; and generating a list of prioritized and biologically relevant alternative splicing events based on prediction of the functional impact of the one or more alternative splicing events. In some embodiments, the semi-supervised or supervised machine learning algorithm comprises: a random forest, Bayesian model, a regression model, a neural network, a classification tree, a regression tree, discriminant analysis, a k-nearest neighbors method, a naive Bayes classifier, support vector machines (SVM), a generative model, a low-density separation method, a graph-based method, a heuristic approach, or a combination thereof. In some embodiments, the machine learning algorithm is trained with a training set, each data point of the training set comprising a feature of the plurality of features, and a label, the label being positive, negative, and unlabeled. In some embodiments, the training set comprises of no less than 50 training data points. In some embodiments, the plurality of features comprises one or more categories of features selected from: RNA-based features, protein domain features, evolutionary features, mutability features, and splicing regulatory features. In some embodiments, the quantitatively estimating probabilities of the one or more alternative splicing events of damaging the protein structures, protein functions, RNA stability, RNA integrity, or biological pathways comprises quantitatively estimating damage caused by: removal of a functional protein domain by alternative splicing; nonsense-mediated decay (NMD) and translation frameshifting (FS) by alternative splicing; mutability of alternative splicing events; weighted closeness centrality of alternative splicing; or a combination thereof. In some embodiments, the annotations comprise one or more selected from: (i) read coverage of every splice junction detected from public data; (ii) frequency and sample types in which a splice site is detected; (iii) likelihood to observe a given alternative splicing variant across a plurality of public samples; (iv) prevalence of alternative splicing events in primary cancers and metastasis, correlation to age, gender and ethnicity, associated survival and relapse rates, and molecular and histological biomarkers; (v) location of alternative splicing events on human genes; (vi) prevalence of alternative splicing events in normal human organs or tissues; (vii) customized features and predictions; and (viii) splicing regulatory interactions (RBP-RNA).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present subject matter will be obtained by reference to the following detailed description that sets forth illustrative embodiments, in which the principles of the invention are utilized, and the accompanying drawings (also “figure” and “Fig.” herein), of which:

FIG. 1 shows an exemplary non-limiting schematic diagram of the systems and methods herein, comprising five exemplary cores: the user interface core, the database core, the compute back end core, the bioinformatics core, and the artificial intelligence (AI) core;

FIG. 2A shows an exemplary non-limiting user login interface;

FIG. 2B shows a non-limiting exemplary user interface for requesting new project(s);

FIG. 2C shows a non-limiting exemplary user interface for selecting datasets for a requested new project;

FIG. 2D shows a non-limiting exemplary user interface for confirming datasets for a requested new project;

FIG. 2E shows a non-limiting exemplary user interface for activating a project;

FIG. 2F shows a non-limiting exemplary user interface for viewing/editing a project, which includes uploaded datasets for SpliceTrap module and uploaded experiment for SpliceDuo module;

FIG. 2G shows a non-limiting exemplary user interface for starting a new experiment by selecting one or more SpliceTrap datasets and one or more case and control datasets;

FIG. 2H shows a non-limiting exemplary user interface for viewing experiment results, which are a list of statistically significant AS changes;

FIG. 2I shows a non-limiting exemplary user interface for customizing, sorting, and filtering of experiment results of AS changes in FIG. 211;

FIG. 3 shows an exemplary non-limiting user hierarchy;

FIG. 4 shows an exemplary non-limiting flow chart for SpliceCore application for input data processing;

FIG. 5 shows an exemplary non-limiting schematic diagram of the set-up, creation, and/or destruction of cluster of computing nodes for the compute back end core;

FIGS. 6A-6C show exemplary non-limiting schematic diagrams of the SpliceTrap module;

FIGS. 7A-7C show exemplary non-limiting schematic diagrams of the SpliceDuo module;

FIG. 8 shows an exemplary non-limiting schematic diagram of the TXdb building module of the compute back end core;

FIG. 9 shows an exemplary non-limiting schematic diagram of feature engineering of the bioinformatics core;

FIG. 10A shows an exemplary non-limiting schematic diagram of the SpliceImpact module of the compute back end core;

FIG. 10B shows an exemplary non-limiting schematic diagram of the SpliceLearn module of the compute back end core;

FIG. 11 shows an exemplary non-limiting schematic diagram of a digital processing device with one or more CPUs, a memory, a communication interface, and a display;

FIG. 12 shows an exemplary non-limiting schematic diagram of a web/mobile application provision system providing browser-based and/or native mobile user interfaces; and

FIG. 13 shows an exemplary non-limiting schematic diagram of a cloud-based web/mobile application provision system comprising an elastically load-balanced, auto-scaling web server and application server resources as well as synchronously replicated databases.

FIG. 14 shows an exemplary non-limiting schematic diagram of the TXdb compilation process comprising extraction of exon duos and exon trios from mRNA molecules present in public repositories or assembled from RNA-seq data.

FIG. 15 shows an exemplary non-limiting graphic representation of the relative number of the four splicing types used in TXdb v1 to indicate the composition of the five annotated categories of TXdb v2 relative to the TXdb v1.

FIG. 16 shows an exemplary non-limiting graphic representation comparing the number of splicing events annotated in the TXdb v1 against other tools and different categories of TXdb v2.

FIG. 17 shows an exemplary non-limiting graphic representation of a reliability score distribution in different TXdb categories.

FIG. 18 shows an exemplary non-limiting graphic representation of training set results wherein the datasets are labeled as positive or negative based on splicing changes in the MFASS dataset.

FIG. 19 shows an exemplary non-limiting graphic representation of predictive feature sets wherein the number of RBPs supported by each of the methods used to infer RPB-RNA interactions is identified.

FIG. 20 shows an exemplary non-limiting image of SRSF2 RT-PCR amplifications products verified by gel electrophoresis to quantify exon inclusion.

FIG. 21 shows an exemplary non-limiting graphic representation of observed intron retention.

FIG. 22A shows an exemplary, non-limiting image of a user interface environment for a user to organize their projects, available in SpliceCore.

FIG. 22B shows an exemplary, non-limiting image of a user interface environment for a user to review project datasets and experiments, available in SpliceCore

FIG. 22C shows an exemplary, non-limiting image of a user interface environment for a user to review the results of their experiment, available in SpliceCore

FIG. 22D shows an exemplary, non-limiting image of a user interface environment for a user to review a splicing event, available in SpliceCore

### DETAILED DESCRIPTION OF THE INVENTION

Reference will now be made in detail to exemplary embodiments of the disclosure, examples of which are illustrated in the accompanying drawings. Wherever possible, the same reference numbers will be used throughout the drawings and disclosure to refer to the same or like parts.

Constitutive RNA splicing is the process of intron removal and exon ligation of the majority of the exons in the order in which they appear in a gene. Alternative splicing (AS) is a deviation from constitutive RNA splicing, in which certain exons are skipped during the ligation step, resulting in various forms of mature mRNA—AS variants. AS allows for greater RNA and protein diversity.

Many human diseases can be caused by aberrant splicing changes, leading to the expression of toxic mRNA isoforms. According to the Human Gene Mutation Database, up to a third of all disease-causing mutations and half of synonymous cancer-driver mutations impair the splicing of crucial genes. Approximately 370 rare genetic disorders are caused by aberrant splicing. For example, mutations in Splicing Factors (SFs) such as U2AF1, ZRSR2, SRSF2 and SF3B1 are recurrent in about 45-85% of patients with myelodysplastic syndrome (MDS). Other examples are amyotrophic lateral sclerosis, retinitis pigmentosa, Huntington's disease, Alzheimer's disease, cystic fibrosis, familial dysautonomia and spinal muscular atrophy (SMA). The recent approval of the drug SPINRAZA® (nusinersen) for treating SMA presents solid evidence that aberrant splicing manipulation can result in innovative therapies to treat genetic disorders.

Up until the introduction of next-generation sequencing in 2007, the main obstacle to high-throughput splicing analysis was the lack of convenient technology platforms like RNA-seq. Before that, the transcriptomics market was dominated by microarray technology. However, only a few microarray platforms may be suitable for exon-level analysis (e.g., exon arrays). These platforms can be expensive and complex in comparison to gene-level microarrays that are not able to detect splicing events at all. The systems and methods provided herein may advantageously allow detection of aberrant splicing events through exon-level RNA-seq analysis. In addition, the significant decrease in the cost of sequencing and the accumulation of public data repositories may advantageously allow discovery of novel and potential aberrant splicing events thereby facilitating drug target discovery and validation.

One advantage of the systems and methods herein is the exon-centric approach to RNA-seq analysis and transcriptome interpretation, replacing the commonly used gene-centric approach for full-transcript assembly and gene expression quantification. Although diseases caused by splicing-affecting mutations are common, aberrant splicing events can be difficult to identify using the commonly used gene-centric approach. The systems and methods provided herein can be highly sensitive in detecting low-abundance aberrant mRNA isoforms and utilize artificial intelligence (AI), e.g., the SpliceImpact module to predict their disease-involvement, the SpliceLearn module to predict the druggability and controllability of splicing events such as aberrant splicing. For example, a gene-centric approach may typically identify differentially expressed genes and then use gene enrichment (e.g., Gene Ontology) for biological interpretation. Although this process could be biologically insightful, it may fail to produce a list of potential drug targets and aberrant splicing events. In some embodiments, the exon-centric approach provided herein first identifies differentially spliced exons, annotates aberrant splicing events based on their recurrence in public data and utilizes machine learning to prioritize the most disease-relevant and druggable exons. Existing technology may offer tools for gene-centric analysis useful for global RNA-seq profiling such as studying pathways activated by disease processes or drug treatments. However, the lack of exon-centric sensitivity and biological interpretation can make it challenging for them to prioritize specific drug targets. In addition, open-source tools for RNA-seq analysis like Cufflinks, DEseq, EdgeR, RMATs and MAJIQ, may only offer basic RNA-seq analysis leaving the need for biological interpretation largely unmet, so users need to devise their own ways to prioritize drug targets and design therapeutics to control them, which is often done manually and can take a long period of time, e.g., several years. The exon-centric approach herein can offer a vertical path to the identification of disease-relevant splicing events, pointing to specific exonic sequences such as RNA-binding protein binding sites to be targeted by small molecules or antisense RNA by using the SpliceCore platform for drug discovery.

An additional advantage of the present disclosure is that the systems and methods herein are developed and validated. In particular, the capacity of specific components of the system/platform to inform drug discovery efforts has been validated experimentally by independent technology.

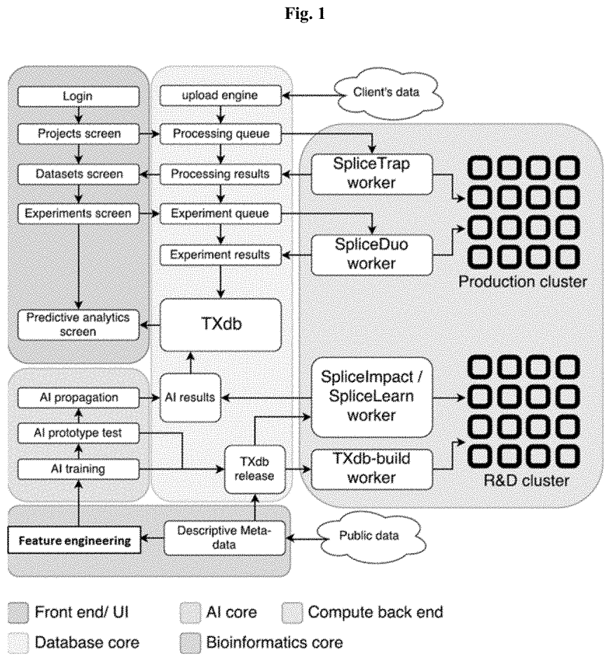

FIG. 1 shows an exemplary schematic diagram of the systems and methods disclosed herein. In this particular embodiment, the systems and methods include 5 core modules that are connected to communicate with others to achieve quantification and analysis of AS. The 5 core modules include a front end/user interface core, an AI core, a TXdb database core, a bioinformatics core, and a compute back end core. Each of the cores can include multiple sub-modules, exemplary sub-modules shown in FIG. 1. In this particular embodiment, a user can log in using the user interface core, request new project(s), and upload datasets for the requested new project. The uploaded datasets can be queued for automatic execution using the SpliceTrap module of the compute back end core. The SpliceTrap module quantifies AS changes to generate results for the user. As an example, the SpliceTrap module generates a plurality of AS values. The quantification results can be reported to the user via the user interface. Using the user interface core, the user may use the SpliceTrap results to perform case/control comparison using SpliceDuo module. The SpliceDuo module may identify statistically significant AS change(s). After SpliceDuo finishes at least a run, the experiment report can be available for viewing at the user interface. The user has the option to combine proprietary data with metadata from the TXdb database core, the bioinformatics core and/or results from the SpliceImpact and SpliceLearn modules. The metadata may provide annotation and mapping reference for the proprietary data of the user. The metadata can also be used by the AI core and the SpliceImpact and SpliceLearn module. With the metadata, the SpliceImpact module can use machine learning to prioritize disease-causing AS changes; and the SpliceLearn module is configured to predict aberrant splicing candidates that can be specific points of therapeutic intervention for the user at the user interface. Such predictive results are available for presentation using the user interface core.

### User Interface

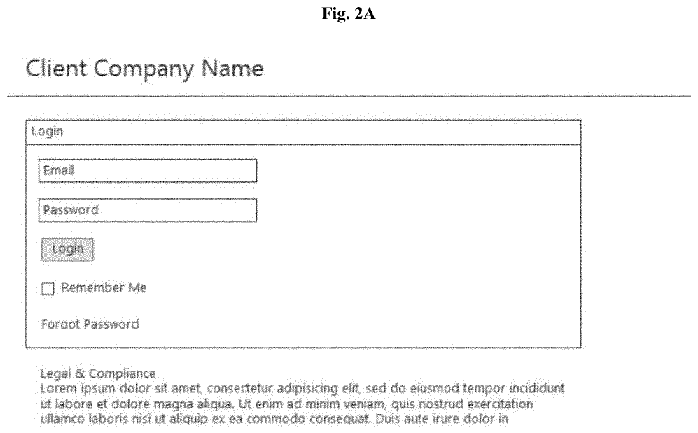

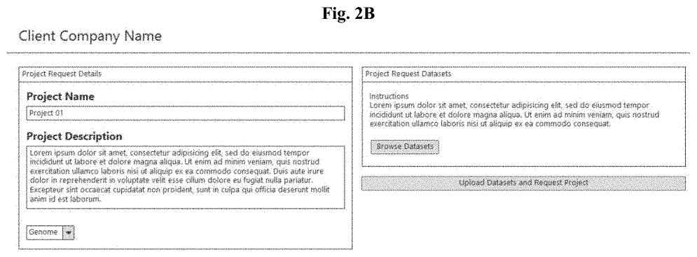

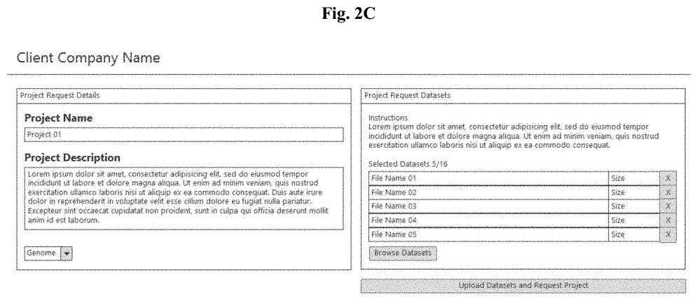

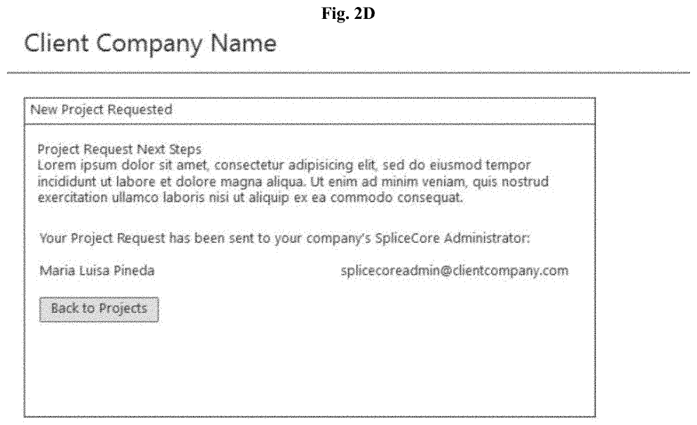

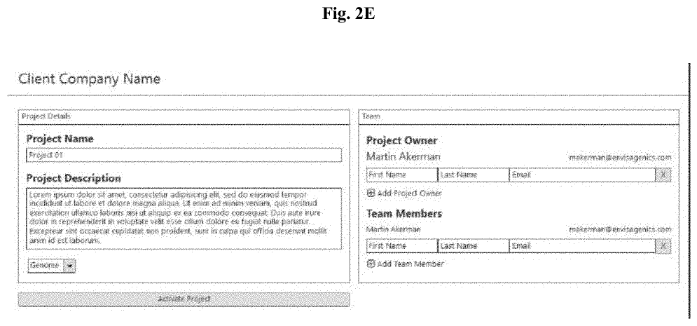

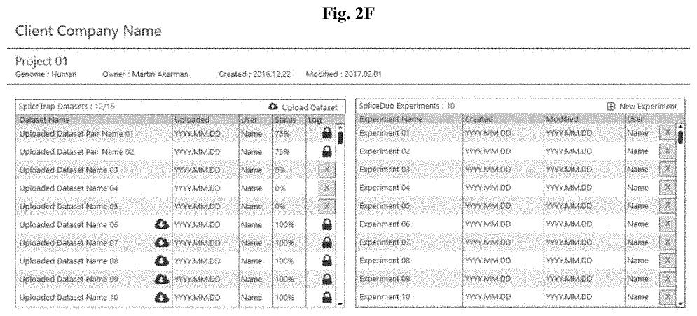

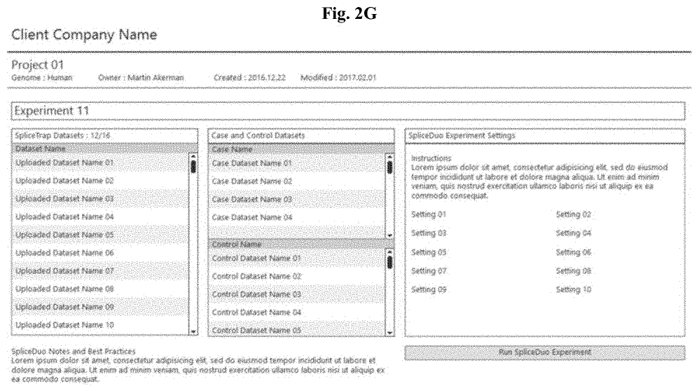

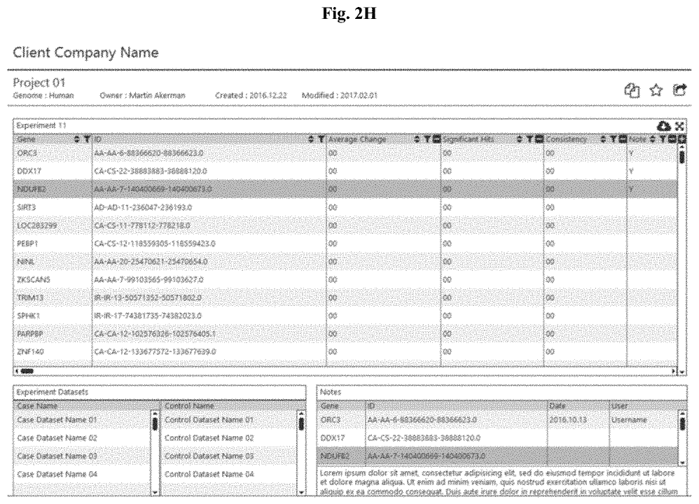

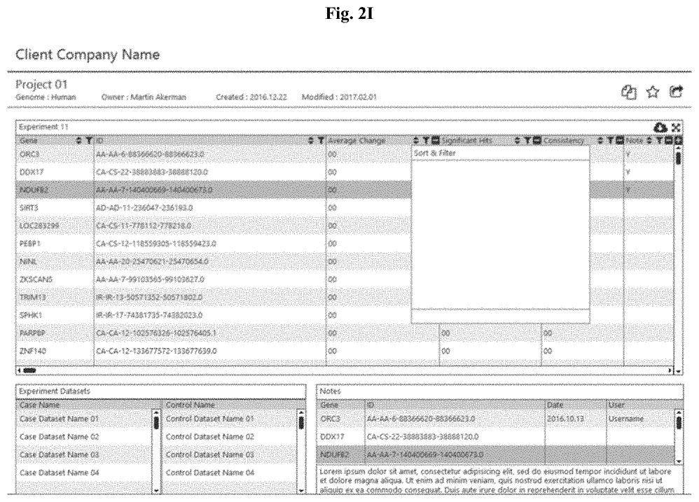

In some cases, the systems and methods herein include a user interface core. As shown in FIG. 2, the user interface core may include a three-tier scheme: (1) project dashboard/screen, for user access management and data upload followed by SpliceTrap analysis; (2) experiment dashboard/screen, where users can select various SpliceTrap outputs to perform case/control comparison using SpliceDuo; and (3) predictive analytic dashboard/screen where users can combine their proprietary data with TXdb metadata and machine learning precalculated predictions (i.e. SpliceImpact and SpliceLearn) for identification of biologically and/or statistically significant AS changes.

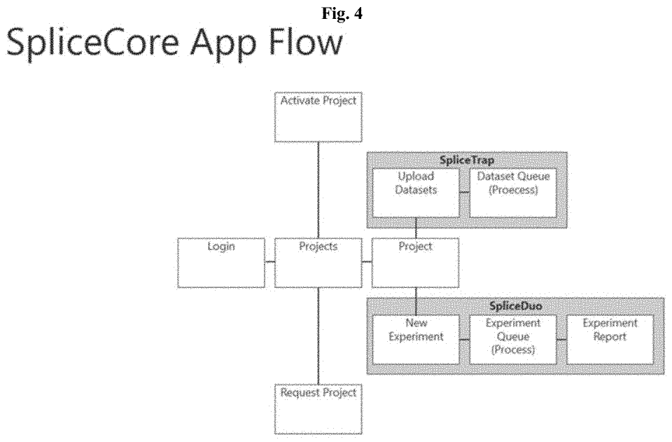

In some cases, the user interface core herein allows a user to use a user-friendly interface for uploading data for quantification/analysis. Such data may include any biological data. Such data may include biological data that can be mapped to genome(s), transcriptome(s), or both. Nonlimiting exemplary biological data is raw RNA-seq data. FIGS. 2A-2I shows nonlimiting exemplary user interface at individual steps of FIG. 4, which allows a user to interactively utilize/edit various functionalities of the SpliceTrap and SpliceDuo modules. For example, after completing multiple SpliceTrap runs, the user can create a SpliceDuo job using the user interface and submit it to be completed as shown in FIG. 2G.

In some cases, the user interface includes interactive functionality that allows viewing, sorting, filtering and merging users' data with TXdb metadata, SpliceImpact/SpliceLearn predictions and SpliceDuo results as shown in FIGS. 2H-2I.

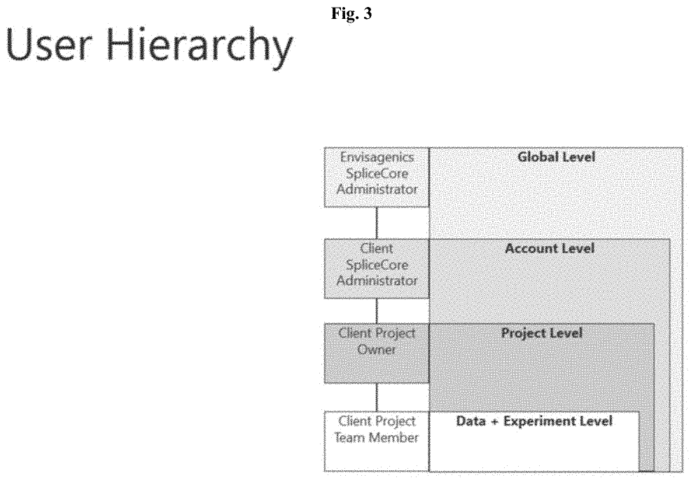

FIG. 3 shows the user hierarchy of different levels of the systems and methods herein. The user project owner may access the projects, datasets, and experiments of the project(s), while the project team member may only access specified datasets and/or experiments of the project(s). The administrator may not only access the users' project information but also account information, and/or information of the system and methods herein that is not provided to the users, for example, the parameters and setting of the SpliceDuo module.

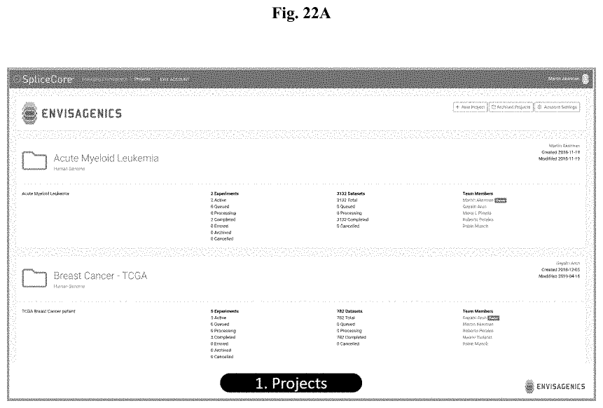

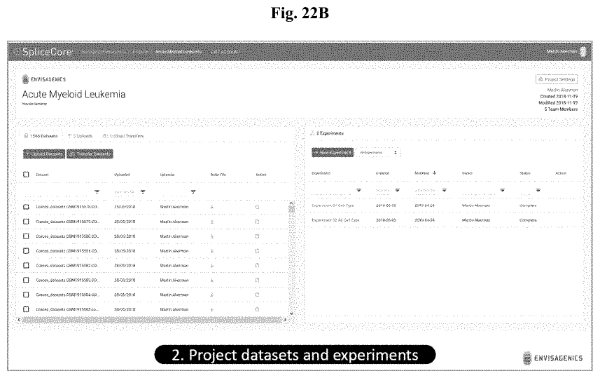

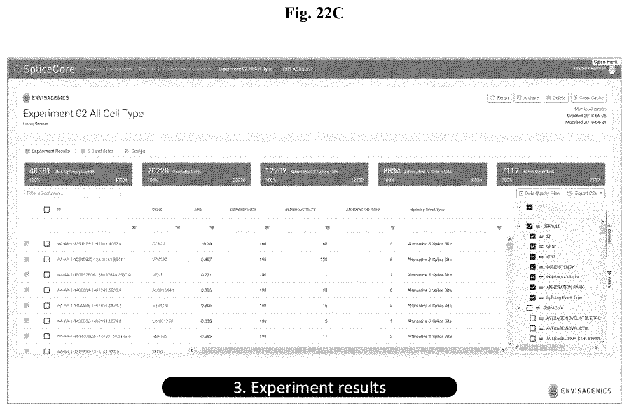

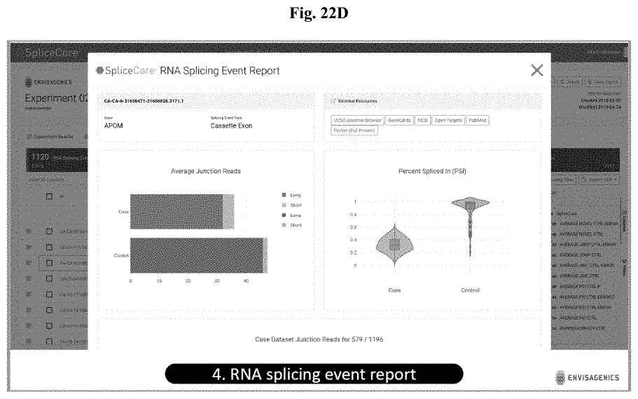

In some cases, the user interface comprising two or more user environments. FIG. 22 shows four exemplary different user environments of the user interface. The first user environment in the top left panel is a Project Dashboard wherein the client's projects can be displayed. Project information can include, but is not limited to, the number of RNA-seq datasets analyzed in the project, the run status of the experiments, as well as admitted users and administrators. The second user environment in the top right panel is Datasets and Experiments. Once RNA-seq datasets are uploaded, they can be analyzed with SpliceTrap and mapped to the TXdb reference transcriptome database. The dashboard can show the analysis process and a link to download data processed by SpliceTrap. The third user environment in the bottom left panel is an Experiments Results interface wherein a table of statistically significant splicing errors is displayed to the user. The columns can include TXdb ID, gene name, dPSI (splicing change), reproducibility (number of case datasets in which the same splicing event was statistically significant), as well as consistency (measurement of agreement between splicing quantification in case datasets.) The fourth user environment in the bottom right panel is a RNA splicing report for the user wherein the user can filter interesting candidates. For each candidate, a series of graphics describing the splicing event can be populated to include such data as splicing levels, read coverage, RNA-seq mapping profiles on the genome, information about disease involvement, tissue specificity, as well as druggability.

### SpliceCore

Disclosed herein are systems and methods for quantifying and analyzing alternative splicing (AS) events. In some embodiments, the systems and methods herein include a platform, e.g., cloud-based platform, to detect, quantify, and interpret AS changes from user input data such as RNA sequence data. Non-limiting examples of input data files includes BAM, SAM, FASTQ, FASTA, BED, and GTF files.

Provided herein is an exemplary platform known as “SpliceCore.” In some embodiments, the SpliceCore platform is equivalent to the compute back end core. In some embodiments, the SpliceCore platform may include one or more modules selected from: the SpliceTrap module, the SpliceDuo module, the SpliceImpact module, the SpliceLearn module and the TXdb build module for building TXdb database.

In some cases, the SpliceCore platform includes one or more of: a software module, an application, an algorithm, a user interface, a memory, a digital processing device, a data storage, a database, a cluster of computing notes, a cloud network, a communications element, and a computer program.

The SpliceCore platform may take as its input user-provided datasets including, but not limited to, biological information that can be mapped to genome(s), transcriptome(s), or both.

In some cases, the SpliceCore platform is configured to provide a stable, scalable, and cost-effective infrastructure to run the SpliceTrap module and/or the SpliceDuo module, for example sequentially, to analyze large amounts of biological data, e.g., RNA-seq data from multiple users simultaneously. In some cases, the platform herein is configured to be adaptable to biopharma bioinformatics workflows, projects' goals and different cloud service providers.

In some cases, the systems and methods herein are configured to use cloud computing, which can advantageously enable parallel distributed computing, cluster computing, compute scalability, training on larger datasets, integration of various data types, and perform deeper search for novel splicing events in reasonable time with lower cost. The alternative to the cloud-based platform herein is to maintain a physical supercomputer. There can be tremendous costs associated with maintaining, protecting and updating such resources. Another benefit of cloud computing can be its scalability. Large cloud computing resources can be temporarily built, utilized, and discarded so that the computing costs vary in direct relation to demand.

FIG. 4 shows a non-limiting exemplary flow chart of the SpliceCore platform. In this embodiment, the user may login to activate a project and upload datasets that are queued for automatic SpliceTrap execution. Under a selected project, the results from SpliceTrap execution can be used in a SpliceDuo experiment that is also queued and executed after user adjustment of experiment parameters. An experiment report can be provided to the user via the user interface, for example, a graphic user interface (GUI).

### SpliceTrap

In some cases, the systems and methods herein include a SpliceTrap module. The SpliceTrap module can include a probability model, e.g., Bayesian model, for the quantification of AS.

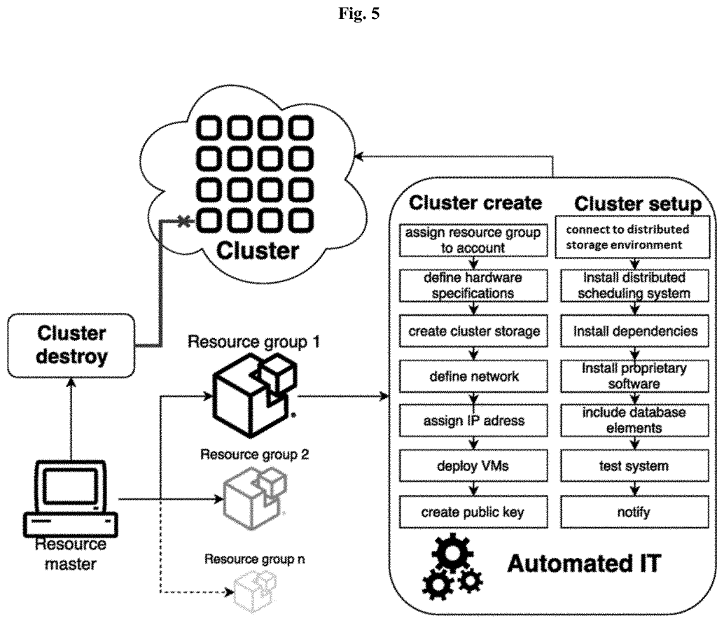

Using the front end, or equivalently, the user interface, the user can select which data file(s), e.g., FASTA/FASTQ, the user wants to upload for analysis by the SpliceTrap module. This upload can create an entry in the SpliceTrap queue which may trigger the creation of the SpliceTrap cluster as shown in FIG. 5. If there is a cluster currently created, a run can be queued. The SpliceTrap pipeline can then process the data and produce its output. After SpliceTrap completes running, the output may be created and uploaded to the user's SpliceTrap results database. The SpliceTrap module can analyze pair-end or single-end transcriptome(s) or genome(s) data for any species for which a TXdb reference can be produced.

In some embodiments, a cluster may include one or more digital processing devices herein, or equivalently, computing nodes. The digital processing devices may or may not be remotely located from the systems and methods herein. In some cases, the devices or computing nodes of the cluster communicate with others in the cluster or the systems and methods herein via a computer network, e.g., a cloud network.

The SpliceTrap module herein, in some cases, includes a software module mapping at least a portion of the user-input information to a database. In some cases, the information comprises biological data related to genome(s), transcriptome(s), or both and/or biological data that can be mapped to genome(s), transcriptome(s), or both. The SpliceTrap module may further include a software module computing a set of data-dependent parameters from the mapped information. In some cases, the SpliceTrap module is configured to perform heuristic approximation to estimate the set of data-dependent parameters. In some cases, the data-dependent parameters from TXdb mapped reads include, but are not limited to, one or more of: fragment size distribution, fragment size distribution model and its parameters, inclusion ratio distribution, inclusion ratio distribution model and its parameters, length of an exon duo or trio isoform, and expression level of an exon duo or trio isoform. The heuristic approximation can result in a significantly decreased runtime than a runtime to compute an exact optimization of the data-dependent parameters. In some cases, the time-consuming estimation of parameters can be replaced with a number of heuristic approximations, resulting in comparable outputs, with very significant run-time reduction. In some cases, the decreased runtime is about 6-40 times less than the runtime to compute the exact optimization of the data-dependent parameters using hardware of similar performance. In some cases, the decreased runtime is no less than 10 times faster than the runtime to compute the exact optimization of the data-dependent parameters using hardware of similar performance. A nonlimiting example of the heuristic approximation is estimating at least one of the set of data-dependent parameters using less than 0.1%, 0.5%, 0.8%, 1%, 2%, 3%, 5%, 6%, 8%, or 10% of the total amount of biological data uploaded by the user. In some cases, the biological data do not include information that is not relevant or can be mapped to genome(s), transcriptome(s), or both. In some embodiments, the biological data can be preprocessed to reduce the size or amount of the biological data without affecting estimation of the data-dependent parameters. For instance, the fragment size distribution (FSD) is a SpliceTrap module parameter based on processing of the entirety of the user input data. Through simulation with 2.8 billion reads from 112 RNA-seq datasets, it is found that minimal sample size for accurate FSD estimation can be 100,000 reads (<1% of the entirety of input data). This can reduce run time from 4.0 min/dataset to 0.2 min/dataset with absolute mean error (MAE) of 0.06%. In some cases, the heuristic approximation includes replacing an inclusion ratio model that is utilized by the SpliceTrap module with a uniformity assumption of inclusion ratio. In some cases, the heuristic approximation includes replacing an inclusion ratio model (IRM) that is utilized by the SpliceTrap module with a data-driven model or mathematical model of inclusion ratio. The inclusion ratio model or other model of similar function can be a time-consuming step to model prior information for SpliceTrap, e.g., IRMs generation for every type of input dataset separately. Replacing IRM with a uniformity assumption can reduce speed to 3.6 min/dataset with 92% of detected AS events showing 0% MAE. In some cases, evaluation of PCR-validated SpliceTrap predictions shows consistency with or without using IRM. In some cases, the heuristic approximation includes using a customized combination for more than one parameters of a thin plate Thin Plate Spline (TPS)-based data smoothing model for identifying one or more statistically significant AS changes, thereby removing the need for iterative calibration of the more than one parameters. SpliceDuo module may iteratively calibrate geometric parameters (e.g., grid size g, number of grids M, and smoothing coefficient k) for its TPS regression model. In some cases, thousands of geometric parameters are simulated on 112 RNA-seq samples and an optimal combination (e.g., g=10, M=100, λ=0.05) can be identified that maximizes AS discovery rate (e.g., ASD-ratio of known vs. predicted AS events), true positive rate (TPR-proportion of reproducible vs. spurious AS events) and/or the amount of detected AS events (N) with run time reduction of 8.8 min/dataset.

In some cases, the SpliceTrap module includes a software module generating a plurality of AS values by applying a probability model, e.g., Bayesian model, to the set of data-dependent parameters. Such plurality of AS values may represent AS changes of the biological data that can be mapped to genome(s), transcriptome(s), or both. In some cases, the AS values are quantitative values that each value can uniquely represent a level of AS changes. In some cases, the AS values herein include exon inclusion ratios and/or percent spliced in (PSI).

In some embodiments, the SpliceTrap module herein quantifies exon inclusion levels in RNA-seq data (e.g., single-end or paired-end RNA-seq data). SpliceTrap module may generate AS profiles for different splicing patterns, such as exon skipping (CA), alternative 5′ (AD) or 3′ (AA) splice sites, and intron retention (IR). It may utilize TXdb database to estimate the inclusion level of every exon as an independent Bayesian inference problem. Unlike microarray-based methods, SpliceTrap may rely on RNA-seq, and therefore it can determine the inclusion level of every exon within a single cellular condition, without requiring a background set of reads to estimate relative splicing changes.

In some cases, the software module quantifying AS is automatic. For efficiency and runtime reduction, the software module quantifying AS may be executed only once for each input dataset of the biological data related to the genome, transcriptome, or both, e.g., a DNA, RNA, mRNA sequence. In some cases, the input dataset includes RNA-seq data from any existing RNA-seq platforms. In some cases, to optimize the efficiency, convenience, and simplicity of the SpliceTrap module, the software module quantifying AS can run to generate AS values without adjustment by the user, e.g., adjustment of parameters of SpliceTrap module.

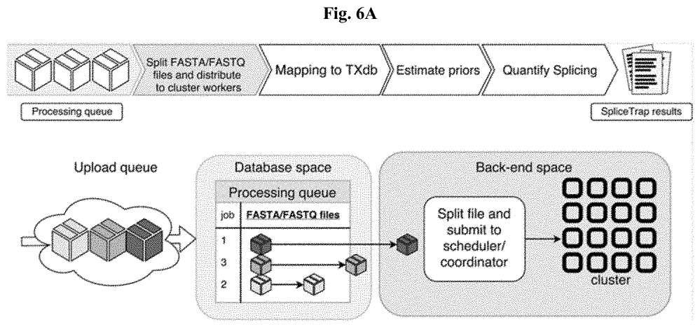

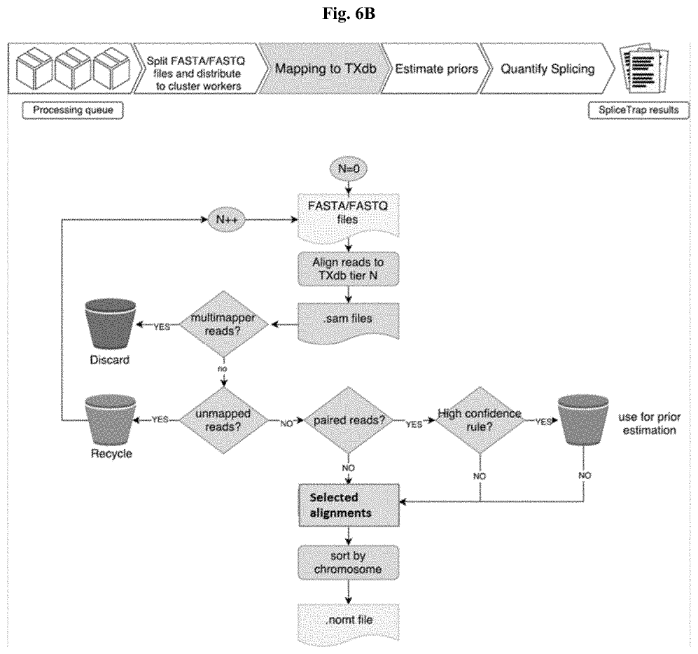

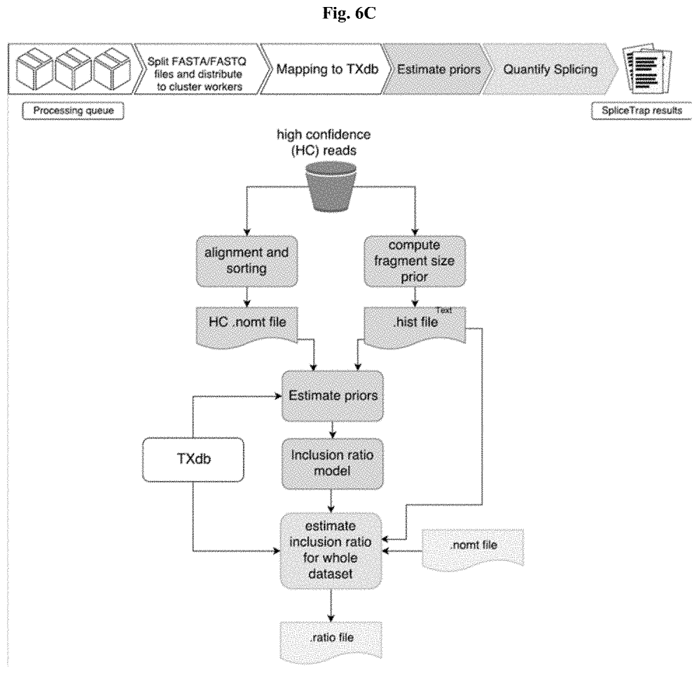

FIGS. 6A-6C show exemplary embodiments of the SpliceTrap module. Referring to FIG. 6A, in a particular embodiment, input files, e.g., RNA-seq data in the form of FASTA or FASTQ file, can be split based on the number of computing cores available on the cluster. Files are split without breaking up reads (e.g., a read is every 2 lines in FASTA and 4 lines in FASTQ). If the input is paired end, the end2 file is split as well.

Referring to FIG. 6B, mapping jobs are done after splitting by mapping the input data to TXdb using an RNA-seq aligner, such as Bowtie or STAR. This may produce a SAM file that contains the TXdb mappings of each read. These alignments are then filtered. Unmapped reads can be removed. If the alignments are to different chromosomes or are far away from each other on the same chromosome, the alignments can be filtered. This can extend to paired end; if the ends are mapped to different chromosomes, the entire read is filtered out. If paired end input is used, the fragment size between the ends is calculated. For each read, the distance between the mappings of gene IDs that exist in both ends is calculated. If this size is consistent for all of the TXdb IDs that are present in both ends, it is added to the fragment size list. These filtered mappings can be split into a file for each chromosome or portion of a chromosome, which can be useful for parallelizing the estimation step.

Referring to FIG. 6C, to estimate the inclusion ratio of each TXdb gene ID, a BED file containing information about IDs can be read. This makes it easy to parallelize by splitting the BED file into multiple pieces. The BED file can be split on a chromosome and each chromosome can be split based on the number of IDs that the chromosome contains. The IDs may also be shuffled to prevent related IDs from ending up in the same file. This is due to the fact that IDs that are near each other usually receive a similar number of mappings and may increase the estimation time of the ID. Thus, shuffling may prevent the IDs that are receiving the most mappings from ending up in the same job. If the input is paired end, the fragment size histogram may be considered.

Subsequently, the file containing the mappings to the chromosome for a particular job is read. For each alignment, the location of the read on the ID is mapped and exon mappings and junction mappings can be counted.

The estimation is then performed on each ID using all of its read pairs. After the first estimation, a model can be created on the inclusion ratios. Only IDs that have coverage of over a threshold, e.g., 10, and a ratio that is not the maximum or minimum acceptable value can be included. To improve the accuracy of the ratios, a histogram of the inclusion ratio model can be used and estimation can be rerun.

Continuing to refer to FIG. 6B, in a particular embodiment, the TXdb database is stratified by at least two levels of reliability, referred as “N”. In this embodiment, reliability refers to the degree at which a given TXdb ID is known and supported by prior data. Prior data can be derived by direct observation of mRNA annotations from the public domain or by using a probability model (e.g., Bayesian model) based on genome-mapped RNA-seq data. In some embodiments, N includes numerical values that indicate reliability of the splicing event(s). For example, N=0 stands for maximum reliability (e.g., well-known and/or characterized splicing events), N>1 refers to varying levels of novelty in TXdb annotations. Levels of novelty can depend on the amount of prior information supporting the existence of those TXdb IDs. After the mapping to TXdb step, transcriptomics reads which remained unfiltered and unmapped are tagged as “unmapped” in the next round of mapping where N=N+1. In some embodiments, except for those reads starting from N=1, among the whole bulk of transcriptomics reads issued in each step with a numerical value for N, only the TXdb IDs that contain reads tagged as “unmapped” at N−1 are moved into the “estimation priors” step. This tagging, recycling, and/or selection step may be key to allow deep exploration of transcriptomics data across a large number of TXdb IDs (e.g., 1 million, 2 million, 5 million or more) at a reduced compute cost and time.

### SpliceDuo

Disclosed herein, in some embodiments, is a SpliceDuo module. The SpliceDuo module can include a software module processing at least a portion of the biological data that can be related or mapped to genome(s), transcriptome(s), or both to identify statistically significant AS change(s). In some cases, the SpliceDuo module applies a regression model, e.g., Thin Plate Spline (TPS) based regression model, to the results calculated from SpliceTrap module, e.g., a plurality of AS values. In some cases, the SpliceDuo module applies a regression model to the biological data that can be mapped or related to genome(s), transcriptome(s), or both. A nonlimiting example of the regression model is a TPS model.

In some cases, the user accesses the SpliceCore front end and creates a new experiment. The user may select which samples the user sets as case and control and determine various experiment parameters. In some cases, the user can only select samples that have been previously processed by the SpliceTrap module. The selected configuration may then be uploaded to the user's database in the experiment table. The experiment event may be uploaded to the SpliceDuo queue. In some cases, the SpliceDuo server is notified that there is an experiment available to be run. A SpliceDuo cluster can be allocated for this experiment based on the number of samples that it uses. The cluster can be created as shown in FIG. 5 and the SpliceDuo experiment begins. After the SpliceDuo experiment is completed, it may automatically upload its results to the user's SpliceDuo results database. The user can then view the report through the front end of SpliceCore or via the user interface core. In some cases, the user also selects to add SpliceImpact and/or SpliceLearn predictions and TXdb metadata to IDs that are in the report. The user may also download the graphs generated by SpliceDuo via the user interface.

In some cases, the systems and methods herein include a software module allowing the user to sort, filter, merge the plurality of AS values representing the AS changes with the information stored in the database, or a combination thereof. This functionality may allow users to rank and prioritize the most important AS changes detected with SpliceTrap and SpliceDuo modules, according to criteria of their choice. It is also possible to customize new metadata, SpliceLearn or SpliceImpact features for example, as requested by biopharma partners.

In some embodiments, the SpliceDuo module includes one or more steps of: data preprocessing, e.g., merging case and/or control datasets; parameter calibration of the regression model to be used, which can be important to avoid over-fitting during the data transformation process; data transformation using a regression model, e.g., Thin Plate Spline (TPS) model; estimation of False Discovery Rates (FDR); and graphic output and/or Duo file output.

In some cases, the SpliceDuo module is configured to identify a set of data-dependent parameters, e.g., parameters of the regression or data regression model including grid size, number of grids, and smoothing coefficient, that maximizes, optimizes an AS discovery rate (ratio of known vs novel AS events), true positive rate (proportion of reproducible vs spurious AS events), a total amount of detected AS events, or a combination thereof to be above a specified threshold. For example, the AS discovery rate or the true positive rate of AS events may be maximized to be above 0.4, 0.5, 0.6, 0.7 or higher.

In some embodiments, case vs control cross-comparisons are performed to identify splicing events that only occur in disease scenarios. Such comparisons can include tens, hundreds, thousands, or larger numbers of datasets. After applying the SpliceTrap and SpliceDuo modules, the SpliceCore platform can identify disease-related splicing events from billions of RNA-seq reads. A high reproducibility filter (i.e. splicing events detected only in a large proportion of the input datasets) is applied to rapidly compare the analyzed data to precomputed public data from The Genotype Tissue Expression project (GTEx), the Cancer Genome Atlas (TCGA) and the Database of Genotypes and Phenotypes (dbGAP) databases. This can be an essential step to confirm aberrant splicing identified in data derived from cancer cell lines or small patient cohorts, with independent data from TCGA cancer patients or a specific tissue from GTEx.

Unlike the large dynamic range of gene-expression values observed in RNA-seq data, exon-inclusion profiles can be restricted to a small range of probability-like values (0 to 1) with a beta (“U”-shaped) distribution. Thus, it can be challenging to assign statistical significance to percent spliced in (PSI) changes using variance of the data (delta_PSI, PSI fold change), or parametric methods such as the t-test for identifying significant outliers. In some cases, non-parametric implementation of Thin Plate Spline (TPS) transformation is used to capture distribution of relative AS changes and assign statistical significance. In some cases, the SpliceDuo module produces a probability density model based on dispersion of AS changes across 2 different conditions. For example, such two conditions can be disease and control, treatment responder and non-responder. In some cases, TPS model(s) is used to estimate false discovery rate (FDR) of each AS change in terms of their pairwise deviation from the density distribution.

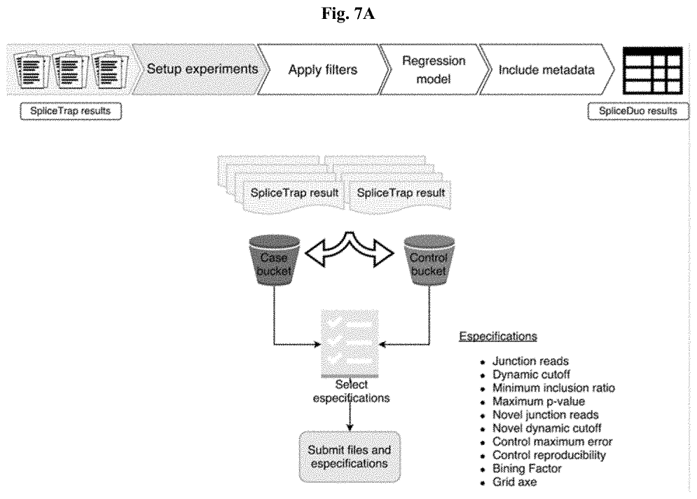

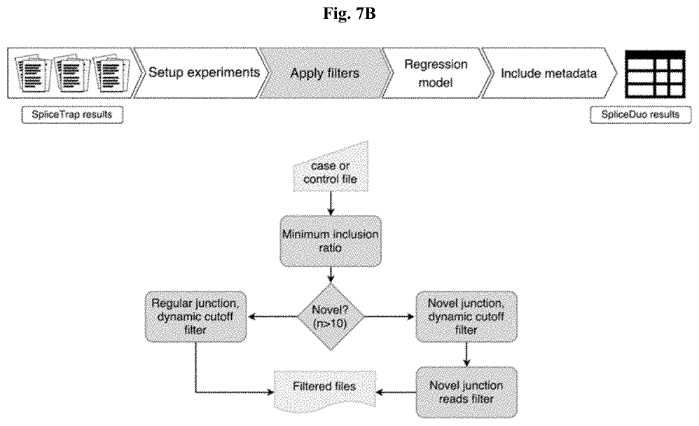

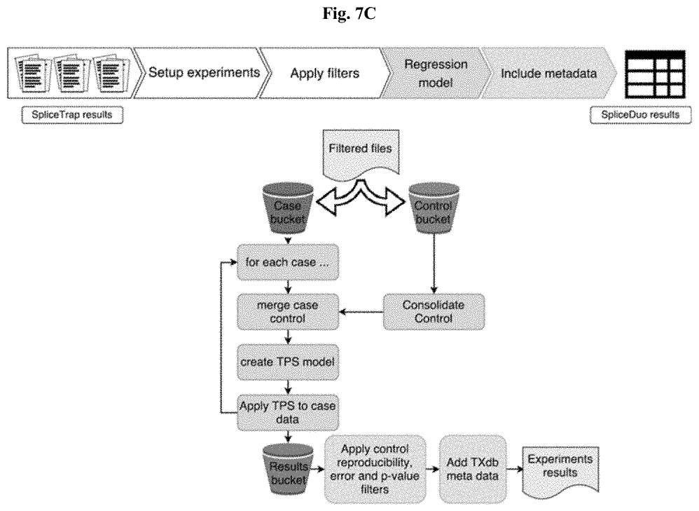

In some embodiments, the SpliceDuo module herein begins by querying the user's SpliceTrap database for the specified samples. Referring to FIG. 7A, in a particular embodiment, the samples are separated to case or control buckets and various specifications can be selected by the user to be used in filtering these samples. Referring to FIG. 7B, the filter is based on multiple cutoffs, including, but not limited to, one or more as specified by the user: minimum inclusion ratio, number of junction mappings, dynamic cutoff based on the inclusion ratio (this may include three levels to choose from), a minimum number of novel reads, maximum p-value, maximum error of control, reproducibility of control, binding factor, and grid axe. The control data can be consolidated by finding the average and average error of: inclusion ratio, long isoform junctions, short isoform junction, and number of novel read mappings. This consolidated control data can then be merged with each filtered case data. This data file can then be split into two files, one for Cassette Exon AS changes and one for all other AS changes.

Referring to FIG. 7C, a Thin Plate Spline regression model is used to smooth the data. A noise regression model is used to assign scores in order to filter out additional IDs. During this process, graphs for each case sample can be created. The data may also be annotated to indicate which genes are associated with each ID that has reached this far in the process. The actual sequence of the ID to the results can be added to produce the final report of the experiment and uploaded to the user's SpliceDuo results database.

### TXdb Database

The TXdb database herein can include a customized database that contains a large number of annotated AS changes derived de novo on public data which are RNA-seq datasets from TCGA, GTEX, and dbGAP, e.g., about 5 million. The size of this customized database can be bigger (about 10 times or more) than comparable open source databases.

In some cases, the TXdb database includes a database configured to allow interrogation through RNA-seq data mapping, wherein each entry of the database may comprise an independent splicing event that is configured to be analyzed by the SpliceCore platform, the SpliceTrap module, and/or the SpliceDuo module.

The TXdb database includes TXdb metadata, which is metadata architecture to rapidly connect partner's proprietary data to public or proprietary clinical or biological data. For every data entry, tens of clinical annotation records are integrated there within, e.g., in 12 different cancer types such as (i) the read coverage of every splice junction detected from public data; (ii) the frequency and sample types in which such splice sites were detected; (iii) the likelihood to observe a given AS variant across a growing number of public samples (e.g., 25,000, 40,000, 100,000 or more); (iv) clinical and cancer-related descriptors of The Cancer Genome Atlas (TCGA) samples such as the prevalence of AS events in primary cancers and metastasis, correlation to age, gender and ethnicity, associated survival and relapse rates, and molecular and histological biomarkers; (v) location of AS events on human genes; (vi) prevalence of AS events in normal human organs and tissues; (vii) SpliceImpact features and predictions (a machine learning classifier that implements Random Forest to predict the biological impact of alternative splicing on protein structure and function); and (viii) SpliceLearn predictions (a machine learning classifier that implements a supported vector machine to predict druggable splicing regulatory sites and/or differentiate between regulated and cryptic splice sites.)

In some cases, TXdb is different from other existing databases; TXdb is also designed to serve as a mapping reference. Existing splicing databases like Appris, are intended for manual interrogation, where users can browse gene names or BLAST sequences of interest. In contrast, TXdb is intended for interrogation through RNA-seq data mapping: each TXdb entry can serve as an independent splicing event analyzed with the SpliceCore platform, which optionally distribute the analysis of a large number of splicing events (e.g., 5 millions) throughout hundreds of computing nodes, optimizing time and cost. In addition, TXdb may have the advantage of being comprehensive, with the inclusion of rare or dubious novel splicing changes. In some cases, a large number of entries in TXdb (e.g., 4.5 millions) are novel splicing changes which cannot be found in existing mRNA databases like ENSEMBL, Refseq and UCSC. Since SpliceCore can run on a scalable cloud computing, resources can be deployed only when necessary, resulting in significant cost savings as opposed to physical computer clusters typically used by universities and pharmaceutical companies which are expensive to maintain. As a result. The SpliceCore platform can carry out a more in-depth exploration of disease-related splicing changes. Other existing databases may lack the capacity to fit compute resources to analytic demand and are not cost-optimized, and also limited in interpretation since they can only detect 20K-300K mRNA isoforms in comparison to the large number of splicing changes in the TXdb (e.g., 5 millions) disclosed herein.

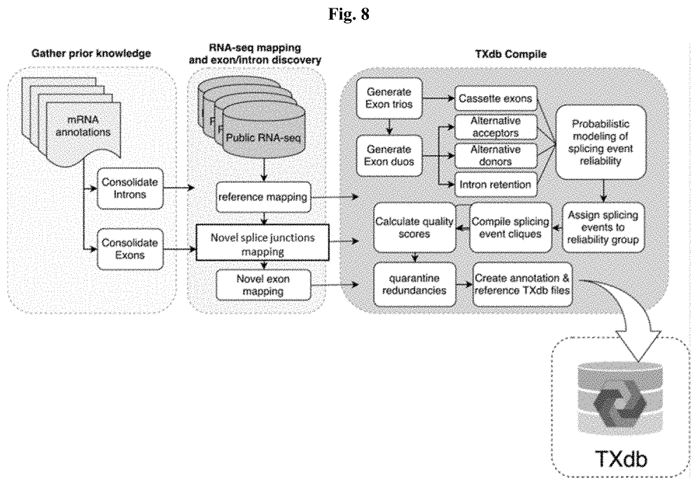

FIG. 8 shows an exemplary embodiment of building the TXdb database using public data and prior knowledge and novel splicing changes. In this particular embodiment, the TXdb database includes annotations and reference TXdb files that can be used as mapping reference(s).

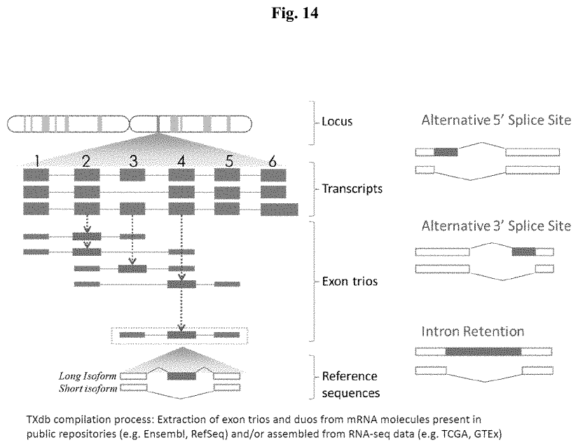

Referring to FIG. 14, in a particular embodiment, a second TXdb database is compiled wherein exon trios are extracted from mRNA molecules present in public repositories. Alternatively, or in combination, mRNA molecules can be derived from sequencing data. Sequencing data may be RNA-seq data from TRGA or GTEx. The TXdb database can comprise the following annotations: cassette exons (CA), alternative acceptors (AA), alternative donors (AD), and intron retention (IR). Cassette exons (CA) can be represented as an exon trio wherein the middle exon is the subject and the flanking exons provide the transcriptomic context with corresponding splice junctions. A software pipeline can be used comprising a STAR aligner, StringTie and differentiation scripts. STAR aligner can be used to detect exon-exon junctions. StringTie can be used for exon trio assembly. Differentiation scripts can be designed to differentiate known from novel annotations and exact the frequency, coverage, and source of the annotations. Frequency can be the number of datasets containing an exon duo or an exon trio. Coverage can be the average, maximum and minimum coverage of the exon duo or exon trio throughout the data. The data source can be the breakdown of diseases and tissue types in which an exon duo or an exon trio was discovered.

Public repositories can include any repository with RefSeq or Ensembl annotations such as NCBI, Ensembl Genome Browser, OMIM, InterPro, Pfam, Prosite, UCSC genome browser, BLAST, etc. Exon duos and/or exon trios can be assigned a reliability score. Reliability scores can be estimated with a scoring function based on Bayesian probability or other statistical and/or machine learning methods that combine one or several variables derived from the RNA-seq data as evidence to support or reject a belief that the exon duo or an exon trio exist in living cells as opposed to being a technical artifact. Example variables to estimate reliability include “Coverage”, which refers to the number of RNA-seq reads supporting the existence of an exon duo or an exon trio and “Frequency”, which is the total number of datasets in which a given exon duo or exon trio is detected.

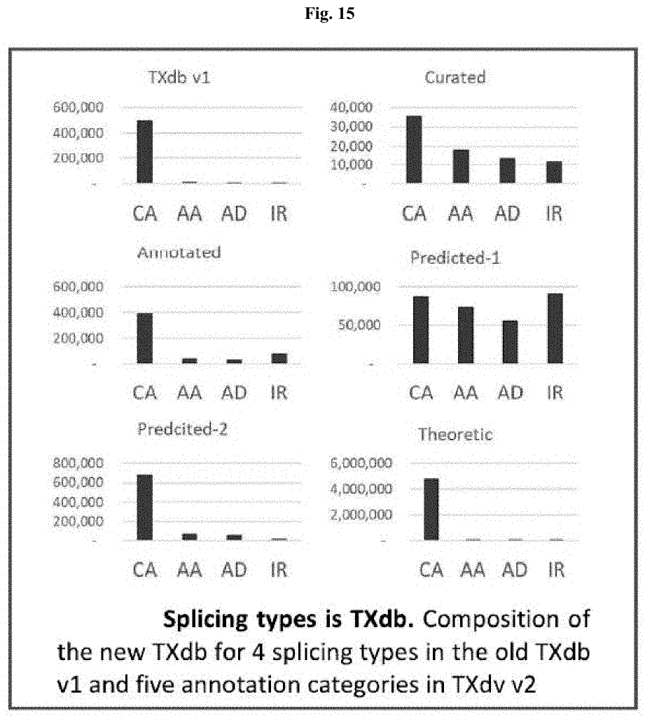

Reliability scores can be calculated by any method known in the art. The reliability score can be used to sort annotations into five different categories. FIG. 15 shows an exemplary graphic representation of the relative contribution of annotations in each of the five categories. One category can be Curated, wherein exon duos and/or exon trios have Ensembl or RefSeq annotations for both inclusion and skipping states. Another category can be Annotated wherein exon duos and/or exon trios with both inclusion and skipping states predicted from Ensembl or Refseq are sorted. A third category can be Predicted-1 wherein exon duos and/or exon trios with both inclusion and skipping states predicted from public repository or sequencing data are sorted. A fourth category can be Predicted-2 wherein exon duos and/or exon trios with either inclusion or skipping states predicted from public repository or sequencing data are sorted. A fifth category can be Theoretic wherein exon duos and/or exon trios likely to exist but with insufficient support evidence are sorted.

### Feature Engineering

In some embodiments, more than one innovative predictive features (e.g., 200 or more) are extracted using public biological databases ranging from protein domain annotations (e.g., Pfam), single nucleotide variants (e.g., ExAc), evolutionary conservation (e.g., PhastCons), CLIP-seq data (e.g., ENCODE), and predicted RNA-binding protein (RBP) RNA interactions (e.g., RBPmap). Such features can be integrated for usage with systems and methods herein, for example, in SpliceImpact and SpliceLearn modules.

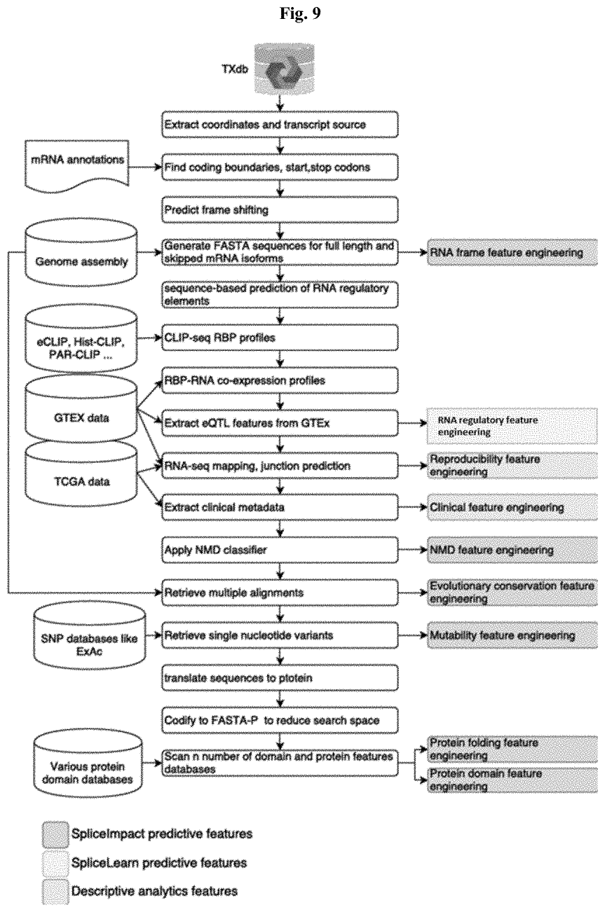

FIG. 9 shows how the features can be extracted from different sources and different types of data. In this embodiment, features can include, but are not limited to, RNA reading frame features (e.g. reading frame size), RNA regulatory features (e.g. splicing regulatory elements), NMD features (e.g. premature stop codons), evolutionary conservation features (e.g. conservation scores), mutability features (e.g. damaging mutation score), protein folding features (e.g. alpha helix probability), protein domain features (e.g. protein domain size), reproducibility features (e.g. frequency in cancer type samples from TCGA). In some embodiments, features disclosed herein are characteristics of the DNA, RNA, mRNA, RNA splicing regulation (e.g., obtained from CLIP-seq data), protein-protein interactions (e.g. yeast 2-hybrid), RNA and protein structure (e.g. mfold predictions), genetic variation (e.g. single nucleotide variants), genetic conservation, (e.g. PhasCons scores), disease pathways data (e.g. Reactome) and custom disease-specific characteristics (e.g. TCGA metadata).

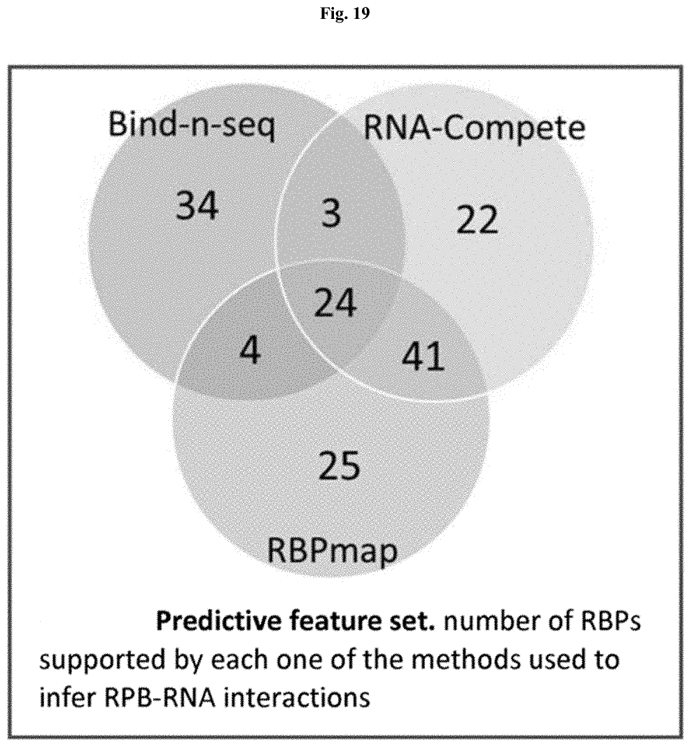

FIG. 19 shows the three methods used by the machine learning (ML) software to infer RBP-RNA interactions from TXdb database version 2 and the number of RBPs supported by each of the methods. The three methods are Bind-n-Seq, RNA-Compete, and RBPmap. A binding score can be estimated for every single nucleotide variant (SNV). The binding scores from each method can be normalized using quantiles or any other statistical methods for scaling and/or standardization such as Z-scores or min-max. The RBPs from each method can be categorized into ontology types, reflecting carious aspects of spliceosomal structure and function as seen in Table 1. The highest quantile score in each ontology can be selected as representative. This data can be used in machine learning feature selection.

TABLE 1
Exemplary table of ontology groups, the number of RBPs in each
ontology and the most predominant RBP families for each of them.

| | | | | Up | Exon | Dn |
|---|---|---|---|---|---|---|
| Class | Ontology | RBPS | Predominant RBP types | Intron | (MW) | Intron (MW) |
| Spliceosome | A_Complex | 62 | SF3 complex, SNRPs | ◯0.01 | ◯0.41 | ◯0.70 |
| structure | B_Complex | 130 | PRPs, SF3 complex, SNRPs | ◯0.05 | ◯0.64 | ◯0.20 |
| | C_Complex | 144 | PRPs, SF3 complex, SNRPs | ◯ [symbol] | ◯0.32 | [symbol] 9.39E−05 |
| | Spliceosome | 32 | HNRNPs, SR proteins | ◯0.19 | ◯0.00 | ◯0.48 |
| | U1_SNRP | 22 | | ◯0.49 | ◯0.16 | ◯0.03 |
| | U2_SNRP | 37 | SF3 complex, SNRPs | ◯0.04 | ◯0.37 | ◯0.58 |
| | U4_U6_SNRP | 7 | PRPs | ◯0.00 | ◯0.27 | ◯0.06 |
| splicing | activators | 13 | HNRNPs, SR proteins | ◯0.02 | [symbol] 3.25E−04 | ◯0.00 |
| Regulation | repressors | 9 | HNRNPs | [symbol] 5.95E−08 | ◯0.98 | ◯ [symbol] |
| | SR_proteins | 15 | SR proteins | ◯9.77E−01 | [symbol] 7.33E−06 | ◯0.47 |
| | hnRNP | 37 | HNRNPs | [symbol] 7.31E−04 | [symbol] 7.12E−04 | ◯0.07 |
| Tissue | rank1_specificity | 15 | | [symbol] 9.37E−04 | [symbol] 4.36E−04 | ◯0.01 |
| specificity | rank2_specificity | 18 | RBMs | ◯0.14 | ◯0.00 | ◯0.11 |
| | rank3_specificity | 20 | RBMs | ◯0.46 | [symbol] 1.39E−05 | [symbol] 4.34E−18 |
| | rank4_specificity | 85 | HNRNPs, RBMs, SR proteins | ◯ [symbol] | ◯0.51 | ◯0.98 |
| evolutionary | essential_proteins | 7 | SF3 complex, SR proteins | ◯0.02 | [symbol] 7.22E−04 | [symbol] 7.20E−05 |
| | conserved_in_yeast | 122 | EIFs, RPLs, RPSs | ◯0.01 | [symbol] 1.26E−05 | ◯0.00 |
| | conserved_in_mice | 146 | EIFs, POLs, RPLs, RPSs, | ◯0.18 | ◯0.32 | ◯0.11 |
| | | | SF3, SNRPs | | | |
| RNA | UAG_motif | 6 | HNRNPs | [symbol] 9.02E−04 | [symbol] 6.05E−07 | [symbol] 1.48E−15 |
| binding | GA_motif | 8 | SR proteins | ◯0.22 | [symbol] 9.92E−06 | [symbol] 1.49E−11 |
| | U_Rich_Motif | 16 | | ◯0.06 | [symbol] 3.45E−04 | [symbol] 2.54E−07 |
| | CG_motif | 4 | | [symbol] 2.55E−04 | [symbol] 1.40E−04 | ◯0.07 |
| | CU_motif | 4 | PPy binding | ◯0.45 | ◯0.00 | [symbol] 1.55E−11 |
| | CA_Motif | 7 | | ◯0.00 | ◯0.01 | [symbol] 1.71E−11 |
| | GUA_motif | 2 | | ◯0.71 | ◯0.20 | [symbol] 1.93E−11 |
| | UG_Motif | 10 | CELFs, RBMs | ◯0.47 | ◯0.33 | [symbol] 5.88E−08 |
| | UAUA_motif | 7 | RBMs | ◯0.43 | [symbol] 2.04E−05 | ◯ [symbol] |
| | GAC_motif | 2 | FMR, FXR | ◯0.01 | [symbol] 8.00E−04 | ◯0.03 |
| | ACA_motif | 3 | | ◯0.25 | ◯0.01 | ◯0.00 |
| | A_Rich_Motif | 6 | HNRNPs | ◯0.01 | ◯0.50 | ◯0.01 |
| | UA_Motif | 6 | HNRNPs | ◯0.94 | ◯0.01 | ◯0.70 |
| | G_Rich_Motif | 9 | ESRPs, HNRNPs | ◯0.12 | ◯0.73 | ◯0.01 |

Table 1: Ontology groups. 153 RBP s were grouped into 32 ontologies representing different aspects of spliceosomal structure and function. We utilized 5 different criteria (Class) to distribute the RBPs. The table shows the number of RBPs in every ontology and the most predominant RBP families for each of them. Of note, a same RBP can be classified to multiple ontologies.We used the Mann-Whitney test to assess, the independent predictive power of each ontology to discriminate between positives and negatives in exons and flanking introns. The table shows the Mann-Whitney P-values. Pie charts are filled at 0%, 25%, 50%, 75%, and 100% as P-values are >1.0E⁻³, >1.0E⁻⁶ and <1.0E⁻⁹, respectively. [symbol]
indicates data missing or illegible when filed

RNA Compete is an in-vitro binding enrichment approach to identify RBP binding preferences using libraries of random k-mers and quantification using microarrays. Binding scores of RMPs to k-mers can be calculated as normalized centered e-scores.

Bind-n-seq is an in-vitro binding enrichment approach to identify RBP binding preferences using libraries of random k-mers and quantification using RNA-seq. Binding scores can be calculated as the ratio between the frequency of k-mers in the RBP-selected pool over the frequency of the input library.

RBP map is a computational tool for the prediction and mapping of RBP position specific scoring matrixes (PSSMs) based on a weighted-weight algorithm which considered the clustering propensity of PSSMs and the overall tendency of regulatory region to be conserved. Binding score can be calculated as Z-scores based on the background distribution of PSSm frequencies.

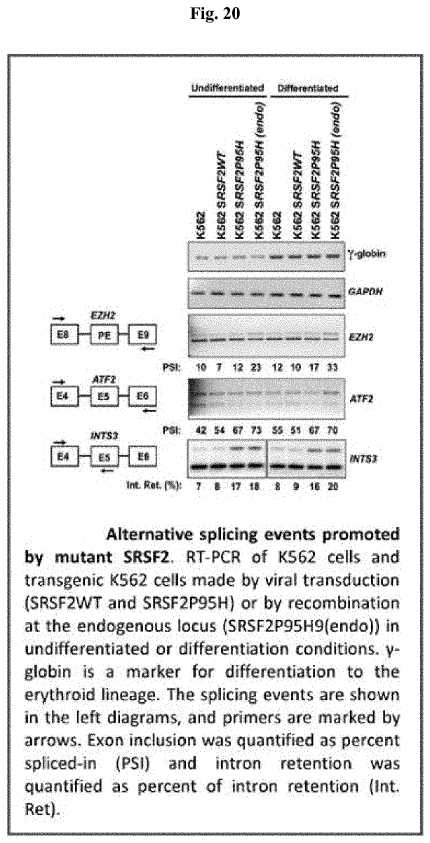

FIG. 20 shows validation of the machine learning (ML) software wherein a myelodysplastic syndromes (MDS) cell differentiation system is used to perform experimental validation of the machine learning (ML) software feature selection using a wild-type (WT) SRSF2 and a cancer-specific SRSF2 mutant. Transgenic knockin human SRSF2 mutant K562 cells can be used along with public RNA-seq data from TSGA acute myeloid leukemia (AML) patients. RNA-seq data from the AML Cancer Genome Atlas was used by the ML software to identify AS events promoted by mutant SRSF2. Hemin can be used to further differentiate transgenic knock-in SRSF2P95H mutant K562 cells to a terminal erythroid lineage since MDS is characterized by defective hematopoietic differentiation. AS events can be validated by RT-PCR. As can be seen in FIG. 20, the splicing events predicted by the ML software were validated by the differentiated transgenic knock-in SRSF2P95H mutant K562 cells.

In some embodiments, the systems, and methods disclosed herein include one or more databases, or use of the same. In view of the disclosure provided herein, many databases are suitable for storage and retrieval of datasets uploaded from user, TXdb metadata, feature information, annotations, AS changes extracted from public data, AS values, quantified or predicted RBP-RNA profiles, one or more software module or computer program of the systems and methods herein. In various embodiments, suitable databases include, by way of non-limiting examples, relational databases, non-relational databases, object-oriented software modules, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### SpliceImpact

The systems and methods herein include a SpliceImpact module. The SpliceImpact module includes a statistical method that integrates protein-protein interactions, RNA and protein structure, genetic variation, genetic conservation, disease pathways data and custom disease-specific features derived from any public or proprietary biological data source, to prioritize biologically relevant AS changes that can potentially cause disease.

In some cases, the SpliceImpact module can include one or more steps selected from: estimating the probability of AS events to down-regulate protein function through nonsense mediate decay (NMD); estimate probability of AS events of damaging protein structures through protein domain deletion; estimating mutability of AS events (the mutability can be determined as the proportion of nucleotides in an exon that when mutated, cause a damaging effect on protein function); mapping AS events with their respective scores in a pathway-pathway network; and outputting list of AS ranked by biological relevance. The protein domains can be retrieved from InterPro database or predicted de-novo using Interpro scan, Pfam, Coils, Prosite, CDD, TIGRFAM, SFLD, SUPERFAMILY, Gene3d, SMART, PRINTS, PIRASF, PRoDom, MobiDBLite, TMHMM and other algorithms to predict functional and structural elements based on primary protein sequences. To estimate the damaging potential of single nucleotide variants (SNV), a combination of functional predictive methods (e.g., SIFT, PolyPhen, Mutation Tester, Mutation assessor, LRT and FATHMM) can be used. Additive damaging score of one or more nucleotides in an exon can be used to prioritize damaging AS events.

In some cases, the systems and methods herein include a software module processing the plurality of AS values with information stored in the database or a second database to identify a plurality of prioritized biologically or clinically relevant AS changes, wherein the software module processing the plurality of AS values with information stored in the database or a second database comprises a supervised or semi-supervised machine learning algorithm, and wherein the information comprises metadata obtained from annotations of a plurality of classes of AS based on public RNA-seq data, CLIP-seq data, genomic data, script data, other biological data or calculated de novo based on DNA, RNA or protein sequences using proprietary or open-source algorithms. In some cases, the systems and methods herein include a software module generating the annotations, wherein the annotation comprises information related to public RNA-seq data and metadata. In some cases, the annotations can also provide mapping reference for the user's input information. In some cases, the systems and methods herein include a software module performing a semi-supervised or supervised machine learning algorithm, wherein the machine learning algorithm takes the plurality of features as an input and outputs a predictive algorithm and/or prediction of impact of AS events on protein structures, protein functions, RNA stability, RNA integrity, or biological pathways. In some cases, the systems and methods herein include a software module processing the plurality of AS values with information stored in a database using the predictive algorithm, prediction (e.g., prediction generated using the predictive algorithm(s) herein or prediction generated using tools external to the systems and methods disclosed herein), and/or the information comprising metadata obtained from annotation of a plurality of classes of AS based on public RNA-seq data. In some cases, the systems and methods herein include a software module generating a plurality of prioritized, and biologically or clinically relevant AS changes based on the plurality of AS values.

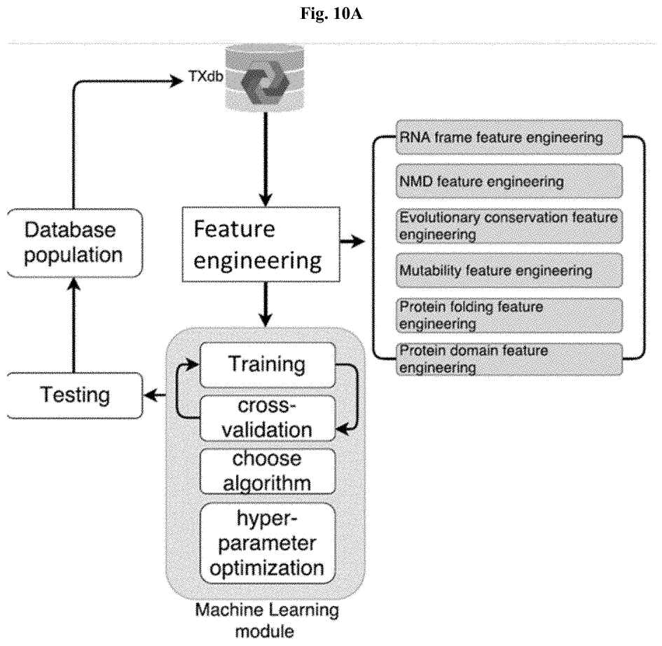

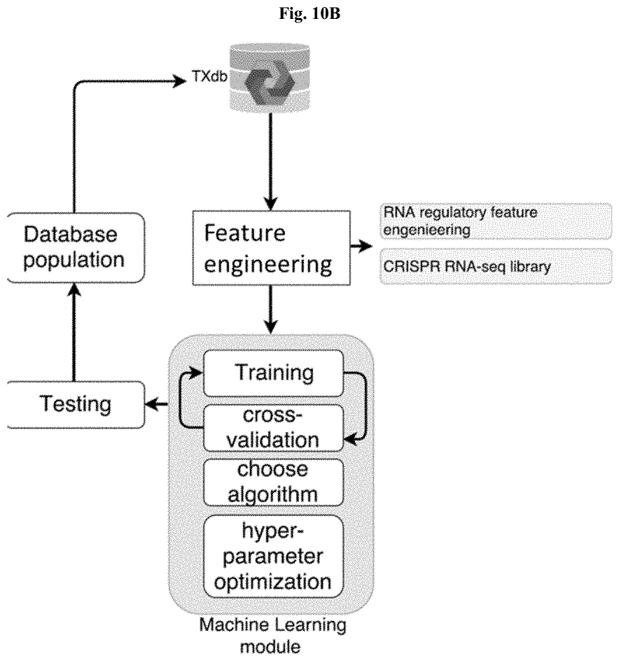

Referring to FIGS. 10A-10B, both the SpliceImpact and the SpliceLearn modules herein use machine learning classifier/algorithm to integrate larger set of predictive features. Non-limiting examples of such machine learning classifier/algorithm includes SVM, random forest, neural networks, logistic regression, and deep learning. In some embodiments, the machine learning algorithm is supervised or semi-supervised to leverage the vast amount of unlabeled AS changes for which no conclusive evidence of functional outcome is known. In some cases, the positive training samples include a number of minor human AS changes (e.g., 943) supported by at least two peptides in PeptideAtlas and not labeled “principal isoform” in the APPRIS database and/or splicing isoforms annotated in Swissprot/ENSEMBL database and supported to result in viable minor splicing events (i.e. low frequency splicing events) as confirmed by TXdb metadata. The positive training set may be separated in two groups of isoforms: minor “skipping” (e.g., 312) and minor “inclusion” (e.g., 631) isoforms, and can be used for training separately.

In some cases, training uses about 100 data points or data sets. In some cases, training uses from about 50 to about 5000 data points.

In some embodiments, multiple descriptive features that can be used for predicting the functional impact of AS events are designed and divided in four categories: 1) RNA-based features, which describe predicted protein length variations due to AS, protein truncation, frameshift and nonsense mediated decay; 2) protein domain features, describing the effect of splicing on protein domains; 3) evolutionary features reporting AS conservation across 45 eukaryote genomes; 4) mutability features, extracted from exome data (Cosmic and ClinVar databases) which assume “important” exons to be less mutated and more included in the mRNA; and 5) custom disease-specific features to adapt the predictions to certain disease scenarios (e.g., gene expression in breast cancer). In some embodiments, the number of descriptive features is dynamically updated. In some embodiments, the number of descriptive features is greater than 200, 300, 400, 500, or more.

In some cases, the machine learning classifier or algorithm can be tested using an independent test set, such as 150 human AS events experimentally confirmed at the protein level by a variety of methods, excluding MS (Hegyi. H. et al., Nucleic Acid Res 2011). The predictability of this particular test set for both exon skipping and exon inclusion models were area under curve of 0.74 and 0.84 respectively.

In addition, the method can be tested with independent disease causing AS events such as 14 known disease-causing AS changes collected from literature. As a result, 6 AS changes were classified as strong negative (i.e. high impact), with scores below 0.2. In addition, another 3 AS events are mildly negative (0.21-0.45). In some cases, the semi-supervised or supervised machine learning algorithm herein comprises: a random forest model, Bayesian model, a regression model, a neural network, a classification tree, a regression tree, discriminant analysis, a k-nearest neighbors method, a naive Bayes classifier, support vector machines (SVM), random forest, deep learning, a generative model, a low-density separation method, a graph-based method, and a heuristic approach.

In some embodiments, the machine learning algorithms herein output algorithm(s) for functional prediction of AS events. The output algorithm(s) may or may not have an explicit or a hidden mathematical expression. The output algorithm(s) may include one or more parameter(s) that can be learned or trained using the machine learning algorithms.

In order to output the algorithm for functional prediction of AS events, a machine learning classifier may include learning the training data, or similarly, a model, or function. For learning, the machine learning algorithm can take training data and/or label as its input data. Learning may be completed when one or more stopping criteria have been reached. For example, a linear regression model having a formula Y=C0+C1x1+C2x2 has two predictor variables, x1 and x2, and coefficients or parameters, C0, C1, and C2. The predicted variable in this example is Y. After the parameters of the model are learned using a machine learning algorithms, values can be entered for each predictor variable in the learned model to generate a result for the dependent or predicted variable (e.g., Y).

A machine learning algorithm herein may use a supervised learning approach. In supervised learning, the algorithm can generate a function or model from training data. The training data can be labeled. The training data may include metadata associated therewith. Each training example of the training data may be a pair consisting of at least an input object and a desired output value. A learning algorithm may require the user to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use a semi-supervised learning approach. Semi-supervised learning can combine both labeled and unlabeled data to generate an appropriate function or classifier.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

A machine learning algorithm may use a feature selection approach. This is a method to optimize the learning accuracy by recursively eliminating the less informative features and keeping the most informative ones. The level of information of every feature can be measured prior to the learning execution (using methods like LASSO, information theory, Shannon entropy) or during the machine learning classification (SVM c-factor, Random Forest feature importance, etc).

A machine learning algorithm may use a transduction approach. Transduction can be similar to supervised learning but does not explicitly construct a function. Instead, tries to predict new outputs based on training inputs, training outputs, and new inputs.

A machine learning algorithm may use a “learning to learn” approach. In learning to learn, the algorithm can learn its own inductive bias based on previous experience.

A machine learning algorithm is applied to training samples to generate a prediction model. A machine learning algorithm may be trained using “positive” vs “negative” or “positive” vs “unlabeled” data. In some cases, each data point of the training set comprises a feature of the set of features, and a label, the labeling being positive, negative, and unlabeled.

In some embodiments, a machine learning algorithm or model may be trained periodically. In some embodiments, a machine learning algorithm or model may be trained non-periodically.

In some embodiments, a machine learning algorithm is interchangeable with a machine learning classifier herein.

### SpliceLearn

The systems and methods herein can include a supervised machine learning classifier or algorithm to differentiate between functional splicing regulatory elements and cryptic splicing regulatory elements of one or more of the AS events thereby predicting controllability of splicing, druggability and/or reversibility of aberrant splicing events. In some cases, the predicting controllability of splicing, druggability and reversibility of aberrant splicing events is configured to be utilized for interpreting splicing events. In some embodiments, the machine learning algorithm(s) under the “SpliceImpact” section are also applicable to the “SpliceLearn” module and other modules or platforms of the systems and methods herein.

To predict specific points of therapeutic intervention, the SpliceLearn module can use machine learning, e.g., supervised or semi-supervised learning, to predict aberrant splicing candidates that could be rescued through induced point mutations (e.g., using CRISPR), use of antisense RNAs (e.g., morpholinos, LNA, ASO), knock down or overexpression of specific Splicing Factors (SF). SF are RNA-binding proteins that regulate both types of splicing: constitutive and alternative. SF mutations can produce widespread aberrant splicing affecting many genes and triggering deregulation of one or more biological pathways. SpliceLearn can train on prior information from splicing profiles, RBP_RNA binding profiles quantified using CLIP-seq data, predicted RBP_RNA binding profiles (e.g., using RBP-map) and/or functional splicing regulatory elements and cryptic splicing regulatory elements (i.e. nonfunctional) or splice sites. This module may implement predictive features extracted from the sequence environment of splice sites as well as RNA-protein interaction profiles from cross-link immunoprecipitation and sequencing (CLIP-seq) of more than 200 SFs, only some of which are publicly available.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

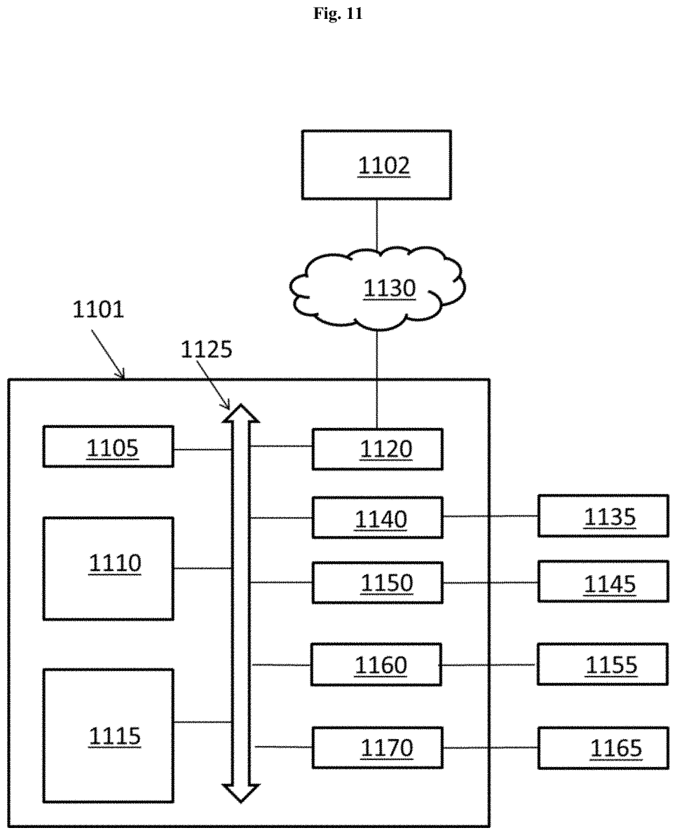

Referring to FIG. 11, in a particular embodiment, an exemplary digital processing device 1101 is programmed or otherwise configured to perform AS analysis and/or quantification and predict biologically significant AS changes. The device 1101 can regulate various aspects of the present disclosure. In this embodiment, the digital processing device 1101 includes a central processing unit (CPU, also “processor” and “computer processor” herein) 1105, which can be a single core or multi core processor, or a plurality of processors for parallel processing. The digital processing device 1101 also includes memory or memory location 1110 (e.g., random-access memory, read-only memory, flash memory), electronic storage unit 1115 (e.g., hard disk), and communication interface 1120 (e.g., network adapter, network interface) for communicating with one or more other systems, and peripheral devices, such as cache, other memory, data storage and/or electronic display adapters. The peripheral devices can include storage device(s) or storage medium 1165 which communicate with the rest of the device via a storage interface 1170. The memory 1110, storage unit 1115, interface 1120 and peripheral devices are in communication with the CPU 1105 through a communication bus 1125, such as a motherboard. The storage unit 1115 can be a data storage unit (or data repository) for storing data. The digital processing device 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the device 1101, can implement a peer-to-peer network, which may enable devices coupled to the device 1101 to behave as a client or a server.

Continuing to refer to FIG. 11, the digital processing device 1101 includes input device(s) 1145 to receive information from a user, the input device(s) in communication with other elements of the device via an input interface 1150. The digital processing device 1101 can include output device(s) 1155 that communicates to other elements of the device via an output interface 1160.

Continuing to refer to FIG. 11, the memory 1110 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 1110 can also include a basic input/output system (BIOS), including basic routines that help to transfer information between elements within the digital processing device, such as during device start-up, may be stored in the memory 1110.

Continuing to refer to FIG. 11, the CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and write back. The CPU 1105 can be part of a circuit, such as an integrated circuit. One or more other components of the device 1101 can be included in the circuit. In some cases, the circuit is an application specific integrated circuit (ASIC) or a field programmable gate array (FPGA).

Continuing to refer to FIG. 11, the storage unit 1115 can store files, such as drivers, libraries and saved programs. The storage unit 1115 can store user data, e.g., user preferences and user programs. The digital processing device 1101 in some cases can include one or more additional data storage units that are external, such as located on a remote server that is in communication through an intranet or the Internet. The storage unit 1115 can also be used to store operating system, application programs, and the like. Optionally, storage unit 1115 may be removably interfaced with the digital processing device (e.g., via an external port connector (not shown)) and/or via a storage unit interface. Software may reside, completely or partially, within a computer-readable storage medium within or outside of the storage unit 1115. In another example, software may reside, completely or partially, within processor(s) 1105.

Continuing to refer to FIG. 11, the digital processing device 1101 can communicate with one or more remote computer systems 1102 through the network 1130. For instance, the device 1101 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Continuing to refer to FIG. 11, information and data can be displayed to a user through a display 1135. The display is connected to the bus 1125 via an interface 1140, and transport of data between the display other elements of the device 1101 can be controlled via the interface 1140.

Methods as described herein can be implemented by way of machine (e.g., computer processor) executable code stored on an electronic storage location of the digital processing device 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

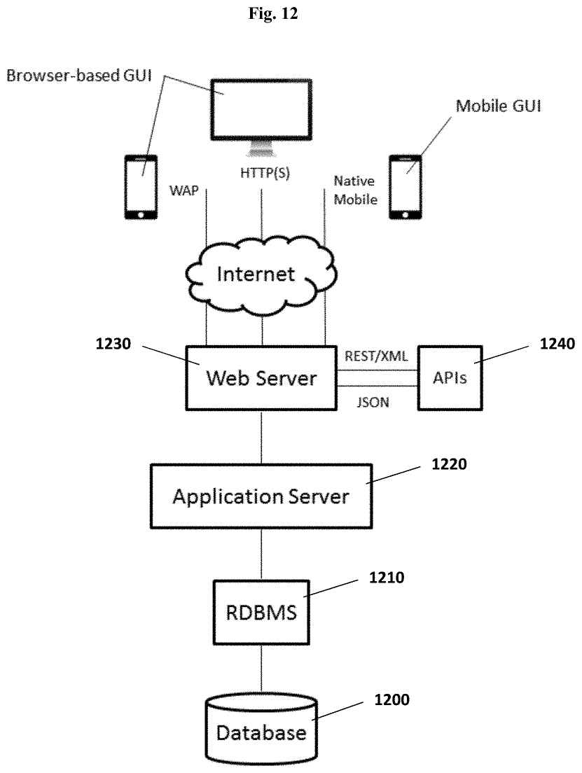

Referring to FIG. 12, in a particular embodiment, an application provision system comprises one or more databases 1200 accessed by a relational database management system (RDBMS) 1210. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1220 (such as Java servers, .NET servers, PHP servers, and the like) and one or more web servers 1230 (such as Apache, IIS, GWS and the like). The web server(s) optionally expose one or more web services via app application programming interfaces (APIs) 1240. Via a network, such as the Internet, the system provides browser-based and/or mobile native user interfaces.

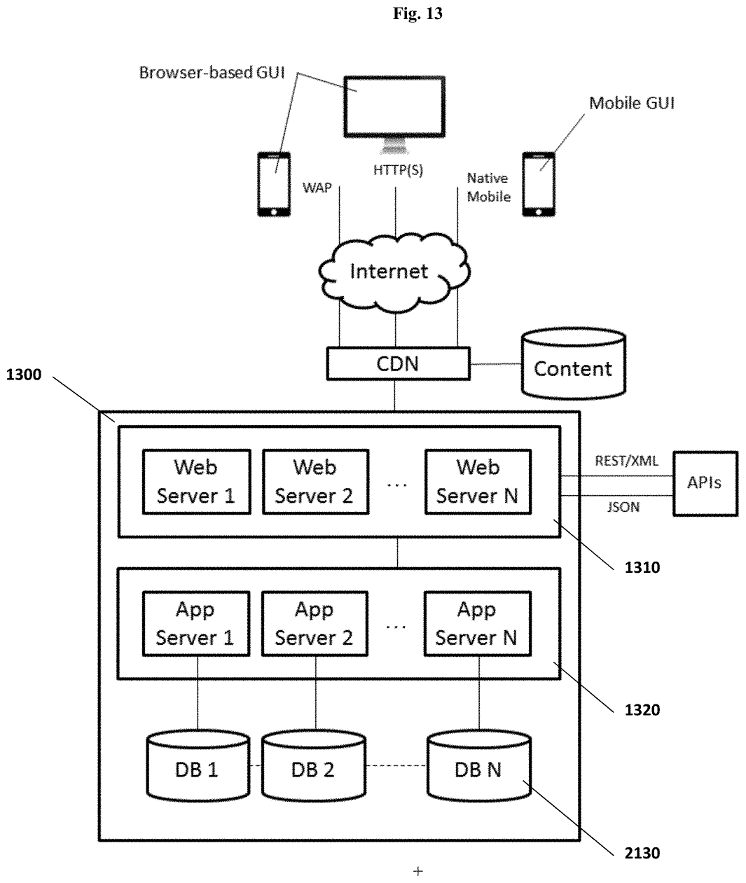

Referring to FIG. 13, in a particular embodiment, an application provision system alternatively has a distributed, cloud-based architecture 1300 and comprises elastically load balanced, auto-scaling web server resources 1310 and application server resources 1320 as well synchronously replicated databases 1330.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable compiled applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Application

Identification of a Disease Condition Associated with a Splicing Factor Mutation

In some embodiments, the platforms, systems, media and methods disclosed herein are applied to medical applications. In one aspect, the proceeding disclosure can be used to identify a disease condition associated with a splicing factor mutation. First, a splicing factor mutation can be identified from an individual's sequencing data. Second, the computer-implemented methods described herein are applied to analyze sequencing data from a database both with and without the splicing factor mutation. An output is then produced containing a list of alternative splicing events promoted by the splicing factor mutation.

Disease conditions can be hereditary or due to exposure to an environmental factor such as radiation, heavy metals, poisons, etc. Disease conditions include but are not limited to cancers, leukemias, disorders of the central nervous system, muscular dystrophies, hormonal disorders and diseases involving immunological disorders such as chronic or abnormal inflammation. Disease conditions may include familial dysautonomia (FD), Spinal muscular atrophy (SMA), Medium-chain acyl-CoA dehydrogenase (MCAD) deficiency, Hutchinson-Gilford progeria syndrome (HGPS), Myotonic dystophy Type 1 (DM1), Myotonic dystophy Type 2 (DM2), Autosomal dominant retinitis pigmentosa (RP), Duchenne muscular dystrophy (DMD), Microcephalic steodysplastic primordial dwarfism type 1 (MOPD1) or Taybi-Linder syndrome (TALS), Frontotemporal dementia with parkinsonism-17 (FTDP-17), Fukuyama congenital muscular dystrophy (FCMD), Amyotrophic lateral sclerosis (ALS), Hypercholesterolemia, and Cystic Fibrosis (CF). Cancers may include but are not limited to bladder cancer, breast cancer, colorectal cancer, gynecologic cancer, cancer of the head, cancer of the neck, hematologic cancer, kidney cancer, liver cancer, lung cancer, pancreatic cancer, prostate cancer, skin cancer, stomach cancer.

Splicing factor mutations include but are not limited to SRSF2, SF3B1, U2AF1, ZRSR2. This also include splicing factors showing aberrant expression in cancer such as members of the SR and hnRNP family, TRA2B, RBFOX1/2, MBNL or any defective RNA binding protein. The database can include public repositories such as the Cancer Genome Atlas, UCSC Genome Browser, NCBI, GTEx, etc. Sequencing data contained by the database can include but is not limited to RNA-seq data and microarray data. Alternative splicing events can include but are not limited to splicing events in BRCA1, BRCA2, EZH2, BIN1, BCL2L1, BCL2L11, CASP2, CCND1, CD44, ENAH, FAS, FGRF, HER2, HRAS, KLF6, MCL1, MKNK2, MSTR1, PKM, RAC1, RPS6KB1, VEGFA, IKBKAP, SMN2, MCAD, LMNA, DMPK, ZNF9, PRPF31, PRPF8, PRPF3, RP9, MAPT, TKTN, TPD-43, LDLR, CFTR, DMD, ATF2, and the gene encoding U4atac snRNA.

Treatment of Disease

The above method can be used to output a list of alternative splicing events promoted by the known splicing factor mutation. The regulatory circuit of the alternative splicing event can then be analyzed for regulatory circuit elements susceptible to alteration or disruption to prevent the alternative splicing event. The affected cells can be sequenced after modification of the regulatory circuit to monitor the presence or absence of the alternative splicing event.

Regulatory circuit elements can be disrupted or modified by methods known to a person of skill in the art. Such methods may include the modification of transcription factors, cis-regulatory elements, inducible transcription factors, constitutive transcription factors, etc. Such methods may include but are not limited to gene silencing by RNA interference or the modification of promoter regions. Methods may further include such components as RNAi, siRNA, CRISPR Cas nuclease, TALENs, zinc finger nuclease, etc.

Identification of Exon Duos and/or Exon Trios Associated with Disease.

In some embodiments, the platforms, systems, media and methods disclosed herein are applied to medical applications. In one aspect, the proceeding disclosure can be used to identify exon duos and/or exon trios associated with a disease condition. The method can comprise first, receiving disease associated gene sequencing data from a database related to a mutation associated with disease. The database can be a public or a private database. The database can include public repositories such as the Cancer Genome Atlas, UCSC Genome Browser, NCBI, GTEx, etc. Sequencing data can be RNA-seq data or microarray data. The alternative splicing event associated with disease can include but is not limited to the following genes: RAS, HER2, p53, BRCA1, BRCA2, EZH2, BIN1, BCL2L1, BCL2L11, CASP2, CCND1, CD44, ENAH, FAS, FGRF, HER2, HRAS, KLF6, MCL1, MKNK2, MSTR1, PKM, RAC1, RPS6KB1, VEGFA, IKBKAP, SMN2, MCAD, LMNA, DMPK, ZNF9, PRPF31, PRPF8, PRPF3, RP9, MAPT, TKTN, TPD-43, LDLR, CFTR, DMD, ATF2, and the gene encoding U4atac snRNA.

Next, the gene sequencing data can be sorted by annotations using the methods disclosed herein to create a TXdb v2 database. This can include a software pipeline comprising a STAR aligner to detect exon-exon junctions, StringTie to assemble exon duos and/or exon trios and a script to differentiate known from novel annotations by analysis of frequency, coverage and source as described herein. The analysis can be run by parallel computing on a cloud service such as the Microsoft Azure cloud. The deployments can be managed automatically with Ansible and Slurm to process the data queue.

Next, a reference transcriptome is created wherein each exon duo and/or exon trio and associated annotation is sorted into two states: inclusion wherein the three exons are present and skipping wherein the middle exon is absent leaving flanking exons only.

Next, a reliability score is applied to each exon duo and/or exon trio and associated annotation using the frequency and coverage of known exon duos and/or exon trios from a database such as Ensembl or RefSeq. A Bayesian-based reliability score can be assigned to every exon duo and/or exon trio using as prior information the frequency and coverage of known exon duos and/or exon trios from databases such as ENSEMBL and RefSeq. The reliability can be calculated as P(R|D)=P(D|R) P(R)/P(D) where R is the probability that the annotation is reliable and D the evidence of reliability. The prior P(R)=P(F≥f|R)P(C≥c|R) is the probability that a given splicing event is observed with a minimum frequency (F) and coverage (C) in the GTEx and TCGA data. P(D|R)=P(F∩C|R) is estimated empirically from Ensemble and RefSeq annotations. The predictor prior can be estimated as P(D)=P(D|R=1)+P(D|R=?) Where R=? is the unknown reliability of unlabeled data and P(F∩C|R)=? is calculated from newly predicted annotations.

Next, the reliability score and whether the exon duo and/or exon trio is in a skipping or inclusion state are used to identify exon duos and/or exon trios as one of five categories. The categories are curated, annotated, predicted-1, predicted-2, or theoretic. Curated includes those exon duos and/or exon trios with annotations for both inclusion and skipping states. Annotated includes exon duos and/or exon trios with either inclusion or skipping states. Predicted-1 includes exon duos and/or exon trios with both inclusion and skipping states predicted from the database. Predicted-2 includes exon duos and/or exon trios with either inclusion or skipping states predicted by the database. Theoretic includes exon duos and/or exon trios likely to exist but with insufficient support evidence. The Predicted categories are output as identifications of novel exon duos and/or exon trios associated with disease.

### EXAMPLES

The following illustrative examples are representative of embodiments of the software applications, systems, and methods described herein and are not meant to be limiting in any way.

### Example 1—CASC4 Exon 9 Discovery

A competitive study published in Breast Cancer Research Treatment uses the open source program MISO to look for AS and validated 4/20 candidates by RT-PCR. In comparison, the systems and methods herein are used to validate 113/155 AS events by RT-PCR. The systems and methods herein identify one of these aberrant splicing events (CASC4 exon 9) as a potential anti-cancer target, as opposed to none by the competitor's software. CASC4 exon 9 is experimentally shown to inhibit apoptosis and increase proliferation as part of the MYC pathway. Before CASC4 exon 9 was singled out as oncogenic using the systems and methods herein, the gene was mentioned only twice in the literature, demonstrating the high innovative value of this discovery using the systems and methods herein.

### Example 2—Construction of a Comprehensive Knowledgebase with Structures AS Information Extracted from Public Data Repositories

A second version of the TXdb database was constructed with alternative splicing information from public data repositories and run to identify novel exon trios. The first version of the TXdb database contains annotations for four different splicing types: cassette exons (CA), alternative acceptors (AA), alternative donors (AD) and intron retention (IR). Every CA is represented as an exon trio where the middle exon is the subject and the flanking exons provide the transcriptomic context with corresponding splice junctions. The concept exon trio was adapted to match the other splicing types (FIG. 14). To identify novel exon trios, a software pipeline was built using STAR aligner to detect exon-exon junctions, String Tie for exon trio assembly, and in house scripts to differentiate known from novel annotations and extract the frequency (number of datasets containing that exon trio), coverage (average, maximum and minimum coverage of the exon trio throughout the data) and source (breakdown of diseases and tissue types in which the exon trio was discovered). Analysis was run in parallel using parallel computing on the Microsoft Azure cloud, and managed automatic deployments with Ansible and Slurm for processing queues. To compile the new TXdb, the RefSeq (GRCh38.p12) and Ensemble (GENCODE v28) annotations were updated first, adding a total of 180,167 publicly known exon trios to the database. In TXdb v2 13,512 annotations from deprecated public records were removed. Next, RNA-seq data from 1,256 TCGA breast cancer (BRCA) and 10,491 GTEx datasets from 31 post mortem tissues were analyzed to identify known and novel tissue-specific splicing events. To prepare the reference transciptome, each exon trio was represented in two potential states: (1) Inclusion, where the three exons are present, and (2) skipping, where the middle exon is absent leaving flanking exons only. In total, 5,980,591 inclusion and 646,405 skipping events were observed in the data.

A Bayesian-based reliability score was assigned to every exon trio using as prior information the frequency and coverage of known exon trios from ENSEMBL and RefSeq. The reliability was calculated as P(R|D)=P(D|R) P(R)/P(D) where R is the probability that the annotation is reliable and D the evidence of reliability. The prior P(R)=P(F≥f|R)P(C≥c|R) is the probability that a given splicing event is observed with a minimum frequency (F) and coverage (C) in the GTEx and TCGA data. P(D|R)=P(F∩C|R) is estimated empirically from Ensemble and RefSeq annotations.

Finally, the predictor prior was estimated as P(D)=P(D|R=1)+P(D|R=?) Where R=? was the unknown reliability of unlabeled data and P(F∩C|R)=? was calculated from newly predicted annotations. This model was used to sort the annotations into five different categories: (1) Curated: Exon trios with Ensemble or RefSeq annotations for both inclusion and skipping states; (ii) Annotated: Exon trios with either inclusion or skipping states in Ensemble or RefSeq; (iii) Predicted-1: Exon trios with both inclusion and skipping states predicted from TCGA and/or GTEx; (iv) Predicted-2: Exon trios with either inclusion and skipping states predicted from TCGA and/or GTEx; (v) Theoretic: Exon trios likely to exist but with insufficient support evidence.

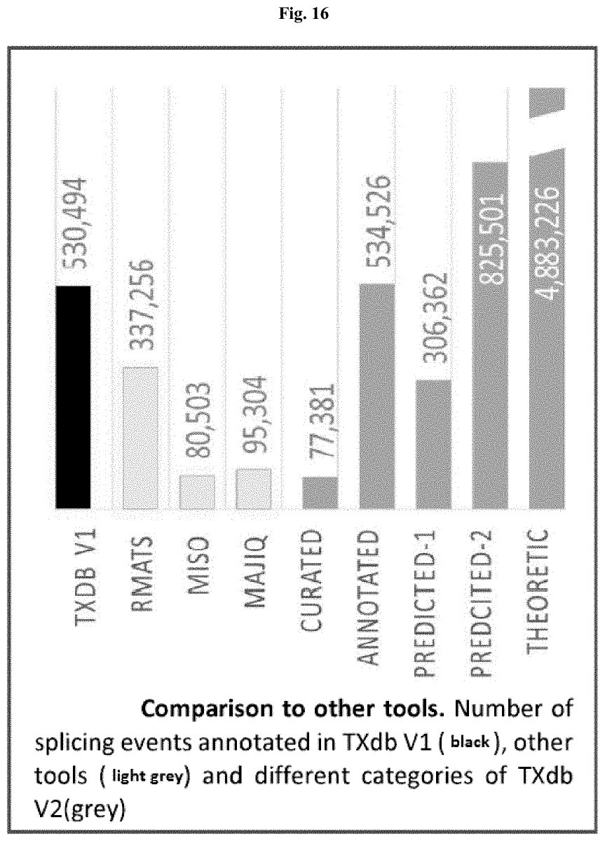

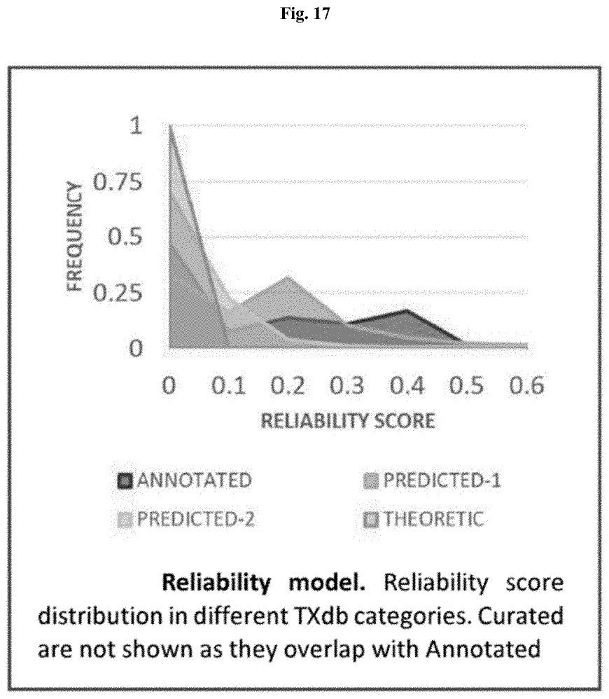

Results: The new TXdb v2 identified a total of 6,626,996 non-redundant splicing events. The Annotated category alone is equivalent in size to the original TXdv v1 and overall the five categories combined amount to >10-fold increase in size. The Curated and Predicted-1 categories concentrate most non-CA splicing events (AA, AD, IR), due to the sorting requirement of both skipping and inclusion isoforms to have similar reliability scores (FIG. 15). When compared to competitive tools, TXdb v2 offers a reference transcriptome at least 20 times bigger than tools such as rMATs, MISO, and MajiQ based on annotation resources available in their respective websites (FIG. 16). The reliability scores calculated with the Bayesian model showed a multimodal distribution with at least four different expectancy groups. Both the curated and annotated categories showed a local maximum reliability of 0.4, while Predicted-1 showed 0.2 Predicted-2 and Theoretic did not have a local maximum but their average scores were 0.05 and 0.0009 respectively (FIG. 17) Interestingly, 143,479 exon trios were observed in at least one BRCA dataset, of which 64,976 belonged to the Predicted group, accounting for 45.3% novel breast cancer specific exon trios in TXdb.

### Example 3: Predicted Regulatory Interactions Between RNA-Binding Proteins (RBPs) and AS Events Annotated in TXdb and Develop a ML-Based Tool for the Identification of Splicing Regulatory Circuits to the Targeted and Modulated by ASO Compounds

Regulatory circuits for the >6 million splicing events in TXdb v2 were identified and annotated. To accomplish this, a ML method trained on high-confidence priors can be applied to the whole TXdb using only RNA-seq data and in-silico RBP binding profiles. Since the number of known and functional ASO binding sites available in the literature is small, single nucleotide variant (SNV) information can be used as a proxy for RBP-specific binding perturbations that alter splicing regulation. It was theorized that any nucleotide sensitive enough to disrupt RBP binding when mutated (e.g. using CRISPR) is likely to respond similarly to ASO blocking. (Cheung and colleagues have recently published a study using a massively parallel splicing minigene reporter for exonic and intronic SNVs, covering 27,733 natural human variants in 2,198 distinct exons. Cheung, R. et al. A Multiplexed Assay for Exon Recognition Reveals that an Unappreciated Fraction of Rare Genetic Cariats Cause Large-Effect Splicint Disruptions Mol. Cell. 73, 183-194. E8 (2019).

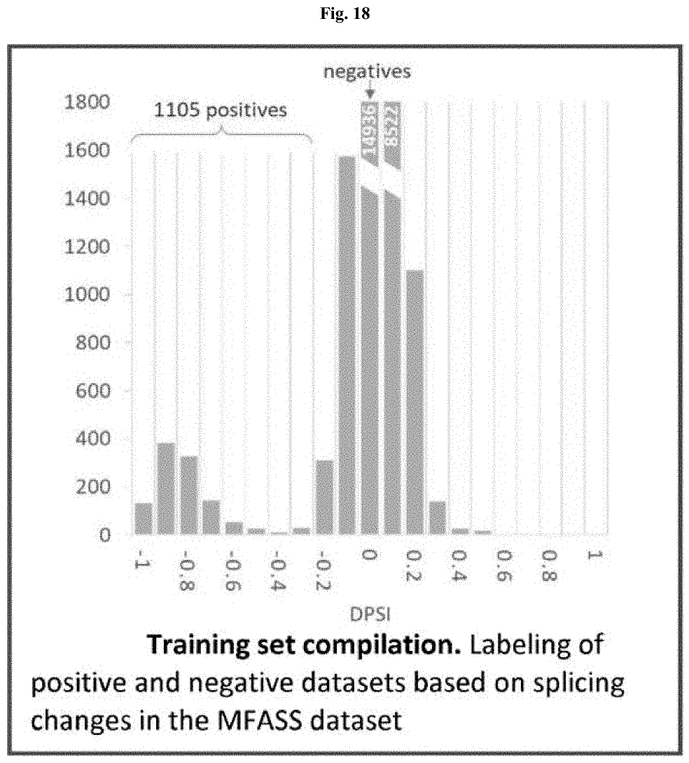

A total of 1,105 SNVs led to a decrease in exon inclusion of at least 25% (ΔPSI≤−0.25), interpreted as potentially removing binding sites for activating RBPs that promote exon inclusion, or conversely creating new splicing repressor binding sites. An additional set of 14,936 SNVs showed no association to changes in splicing (−0.05≤ΔPSI≤0.05), therefore the former was labeled “positive” and the latter was labeled “negative” sets to train a ML classifier that predicts SNVs driving exon skipping (FIG. 18). Three different methods of RBP binding inference based on primary RNA sequence screening were integrated to interpret the effect of SNVs on exon inclusion and to design ML predictive features:

(i) RNA-Complete: In vitro binding enrichment approach to identify RBP binding preferences using libraries of random k-mers and quantification using microarrays. Binding scores of RBPs to k-mers were calculated as normalized centered e-scores.

(ii) Bind-n-seq: Like RNA-complete, except that it uses RNA-seq instead of microarray to estimate the abundance of enriched k-mers. Binding scores were calculated as the ratio between the frequency of k-mers in the RBP-selected pool over the frequency of the input library.

(iii) RBPmap: A computational tool for prediction and mapping of RBP position specific scoring matrixes (PSSMs) based on the weighted-rank algorithm which considers the clustering propensity of PSSMs and the overall tendency of regulatory region to be conserved. The binding scores are calculated as Z-scores based on the background distribution of PSSM frequencies. For every SNV, binding scores were estimated for a total of 153 RBPs covered by at least one of the three methods (FIG. 19) and normalized the three scoring functions using quantiles. Next, to design intuitive and biologically-relevant predictive features while reducing the dimensionality and sparsity of the RBP matrix, RMP subsets were integrated into 32 ontology types, reflecting the various aspects of spliceosomal structure and function (Table 1). Different RMPs in a same ontology were combining by selecting the highest quantile score as representative, and then summing scores across the three methods to reward proteins with higher evidence support. The intuition behind this scoring function is that commonly, a single RBP predominantly occupies a splicing regulatory motif, even if it needs to outcompete other RMPs (i.e. other members of a given ontology). Using this dataset, preliminary feature selection was performed in preparation for ML training and testing.

Results: The Wilcoxon test was utilized to assess the predictive power of each individual ontology when comparing the Positive (i.e. SNVs that promote exon skipping) and Negative datasets (i.e. SNVs with no effect on splicing) in three different sequence regions: (i) exonic SNVs, and SNVs occurring (ii) in the upstream intron or (iii) in the downstream intron (Table 1). According to this analysis, SNV-mediated removal of exonic SR protein binding sites is a strong predictor of decreased exon inclusion (p<7.33⁻⁶). This aligns with many previous reports describing SR proteins role as splicing activators that bind GA-rich exonic sequence enhancers to promote exon inclusion. Accordingly, the exonic activator (p<0.0003) and exonic AG-rich binding motifs (p<9.92⁻⁶) were highly significant. Interestingly, intronic SNVs affected different functions whether occurring upstream or downstream skipped exons. In the upstream sequence flanking the 3′ splice sites, splicing repressors including several members of the hnRNP family, where highly predictive (p<5.9⁻⁸) along with CG-binding RBPs (p<0.00025). A particularly strong set of features was observed in downstream introns close to the 5′ splice site, including proteins present in the spliceosomal C complex (p<9.39⁻⁶), essential RBPs (p<7.2⁻⁵) and RBPs ranked 3 in tissue specificity (p<4.34⁻¹⁸) which is explained by the fact that several RBP such members of the SF3 sub-complex or poly-A binding proteins such as CPEB2, CPEB4, and PCBP1 are essential proteins, members of the spliceosomal C complex, and tend to be ubiquitously expressed throughout tissue types.

### Example 4: Predicted Regulatory Interactions Between RNA-Binding Proteins (RBPs) and AS Events Annotated in TXdb and Establish MDS Cell Differentiation System to Perform Experimental Validation of the ML Software Using WT SRSF2 and Cancer-Specific SRSF2 Mutant

Cancer-specific model cell lines, computational pipelines and biochemical approaches to address the functional significance of specific motifs in regulating cancer-specific AS by promoting RBP-RNA interactions were used. Transgenic knock-in human SRSF2 mutant K562 cells (human myelogenous leukemia cells) and mining public RNA-seq data from TCGA acute myeloid leukemia (AML) patients were used to identify SRSF2 splicing targets in the context of MDS/leukemia.

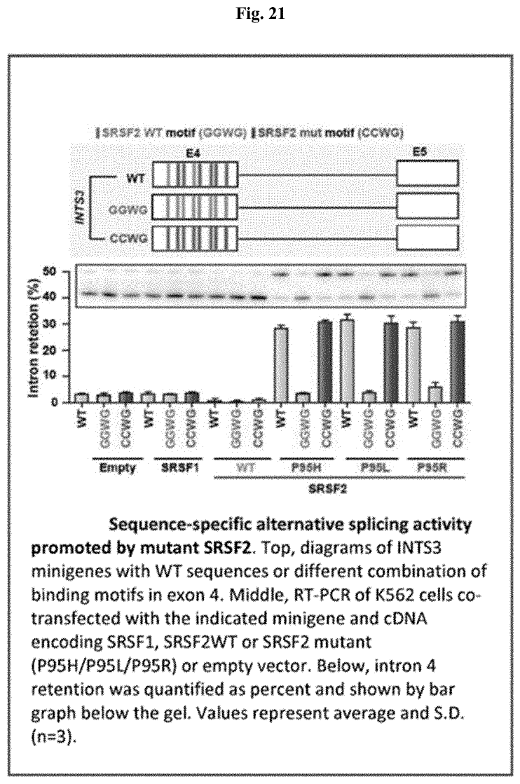

RNA-seq data from the AML Cancer Genome Atlas (TCGA) with or without SRSF2 mutations, to identify AS events promoted by mutant SRSF2 was analyzed. Transgenic knock-in SRSF2P95H mutant K562 cells were used for experimental validation. MDS is characterized by defective hematopoietic differentiations, therefore K562 cells were further differentiated to the terminal erythroid lineage using hemin. Using RT-PCR, several AS events were validated. Among them, a poison exon inclusion event in EZH2 and an exon inclusion event in ATF2, were previously reported. Consistent results were obtained, as seen in FIG. 20. These results validated the suitability of the model cell line and experimental system. In addition, a novel AS event INTS3 in TCGA-AML RNA-seq data was identified. Retention of two consecutive introns (introns 4 and 5) were found in INTS3 which generate premature termination codons. It was predicted that the premature termination codons target the mRNA for nonsense-mediated mRNA decay. INTS3 (Integrator Complex Subunit 3) is a member of the Integrator complex, which play important role sin both transcription initiations and the release of paused RNA Polymerase II. Retention of intron 4 was validated by RT-PCR in SRSF2 mutant cells (FIG. 20). According to recent reports, SRSF2 WT prefers to bind a G-rich motif (GGWG, W=A/U) and SRSF2 mutant prefers to bind a C-rich motif (CCWG). To investigate, whether mutant SRSF2 promotes intron retention in INTS3 in a sequence-specific manner, a minigene reporter spanning exon 4 to exon 5 was generated, including intron 4 (FIG. 21). There are two GGWG motifs and four CCWG motifs in exon 4 (WT minigene). Two additional versions of INTS3 minigenes were generated by mutagenesis harboring either GGWG motifs (GGWG minigene) or CCWG motifs (CCWG minigene) in exon 4. Each of these minigenes was cotransfected with cDNA encoding SRSF2 WT or SRSF2 mutant (P95H/P95L/P95R) in K562 cells and analyzed splicing by RT-PCR. SRSF2 WT showed no activity on intron retention in any of the minigenes. However, SRSF2 mutants promote intron retention for WT and CCWG minigenes, but not for the GGWG minigene. This demonstrated a sequence-specific novel function of SRSF2 WT.

### Example 5—SpliceCore's System Architecture and User Interface

1. Automated back-end deployment and scalability: Automated IT infrastructure was developed to enable automatic platform deployment and compute resource management, allowing the SpliceCore platform to be easily “cloned” in independent Azure accounts for our users. This development ensures complete isolation of proprietary datasets in compliance with user data policies who own the Azure account. Therefore, the data does not leave the organization, the software is linked to the data, and the user maintains the ability to manage the type and amount of computing resources including storage and virtual machines to adapt run time and cost to each project requirement.

Automatization of high-performance computing clusters using Terraform and Ansible: the terraform code created Azure virtual machines, Azure storage containers, necessary disks, security policies and storage containers. Also, Terraform automatically descales or destroys resources once analysis is complete. An Ansible playbook was written to install and configure Slurm for job parallel orchestration, toolsets (e.g. bowtie, samtools), packages and modules (e.g. Python, R) and all the proprietary code to perform splicing analysis and data interpretation with the SpliceCore platform. The engineering tasks of the computing clusters include: (i) Error handling was improved with backend infrastructure and workflow, added email notifications to workflow process on completion or errors. (ii) Cloud data downloads from remote cloud storage environments (e.g. AWS S3) and data upload were refactored. (iii) A PostgreSQL database structure was developed to encapsulate new data points produced by the workflow in SpliceCore reports. (iv) Extraction of data reports from PostgreSQL database server to Azure Database for PostgreSQL services using Azure Redis Cache services was refactored.

2. Front end user interface (UI): SpliceCore's UI is a collaborative environment that allows the exchange of data, information and insight with users. The UI enables upload and analysis of RNA-seq data with our algorithm, connecting splicing quantification results to built-in predictive-analytic tools such as SpliceImpact or TXdb meta-data. An interactive table was developed that allows to data integration in real time as well as graphic visualizations to assist the selection of drug targets and biomarkers. The engineering tasks of the front end user interface include: (i) Design of modern and responsive UI with Bootstrap 4 and Ruby on Rails 5.2.2. (ii) Refactored and increased performance of PostgreSQL databases for project and experiment data. (iii) Improved the performance, scalability and filtering of experiment results table using agGrid and JavaScript. (iv) Added splicing event report data visualizations such as case and control junction reads and GTEx reproducibility using Plot.ly JavaScript libraries. (v) Integrated external web research tools such as UCSC Genome Browser, GeneCards, NCBI, Open Targets, and PubMed. (vi) Increased security with native Mircosoft Azure virtual machine and storage services.

SpliceCore's cloud environment and UI is divided in four environments, as seen in FIGS. 22 A, B, C, D:

(i) Project Dashboard: Displays a list of client's projects and for each one, the number of RNA-seq datasets analyzed in that project, the run status of experiments, admitted users and administrators. Clicking on the project's name launches the datasets and experiments dashboard (FIG. 22A).

(ii) Datasets and experiments: Displays a list of uploaded RNA-seq datasets on the left side and a list of experiments on the right. One RNA-seq datasets are uploaded they are automatically analyzed with SpliceTrap and mapped to our reference transcriptome and database TXdb. The dashboard shows the analysis process and once ready the SpliceTrap outputs (ratio files) become available for experimentation and can also be downloaded. An experiment is a case control comparison between two different groups of RNA-seq data using SpliceDuo. By clicking on the Experiment design button, the user can choose and select RNA-seq datasets to e used ine ach experiment. The experiment status appears on the right side. Once experiments re completed they can be clicked to launch the experiments result dashboard (FIG. 22B).

(iii) Experiments results: this is an interactive table displaying the number of statistically significant differential splicing erros. The default columns display TXdb ID, gene name, dPSI (splicing change), reproducibility (number of case datasets in which the same splicing event was statistically significant) and consistency (a measurement of agreement between splicing quantification in case datasets). In addition, the right pane offers hundreds of additional columns to be added to the output, including precalculated splicing event sin GTEx and TCGA, patient meta data and ApliceImpact results. The columns can be added, removed, sorted and filtered in real time, allowing seamless integration of several datasets. (FIG. 22C).

(iv) RNA splicing report: After filtering of interesting candidates one can click the left blue square associated with every splicing event to visualize a series of graphics describing every splicing event. The visualization included splicing levels, read coverage, RNA-seq mapping profiles on the genome, information about disease involvement, tissue specificity and druggability (FIG. 22D).

Although certain embodiments and examples are provided in the foregoing description, the inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein, A and/or B encompasses one or more of A or B, and combinations thereof such as A and B. It will be understood that although the terms “first,” “second,” “third” etc. may be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

1.-28. (canceled)

29. A computer-implemented system for quantifying functional impact of alternative splicing events on protein structures, protein functions, RNA stability, RNA integrity, or biological pathways comprising: a digital processing device comprising: a processor, an operating system configured to perform executable instructions, a memory, and a computer program including instructions executable by the digital processing device to create an alternative splicing functional impact analysis application, the application comprising a software module for:
(a) generating a plurality of features based on information stored in a database, wherein the information comprises metadata obtained from annotations of a plurality of types of alternative splicing based on public RNA-seq data or other biological data;
(b) obtaining one or more alternative splicing events;
(c) quantitatively estimating probabilities of the one or more alternative splicing events of damaging the protein structures, protein functions, RNA stability, RNA integrity, or biological pathways based on the plurality of features;
(d) applying a supervised or semi-supervised machine learning algorithm to predict the functional impact of the one or more alternative splicing events based on the estimated probabilities; and
(e) generating a list of prioritized and biologically relevant alternative splicing events based on prediction of the functional impact of the one or more alternative splicing events.

30. The computer-implemented system of claim 29, wherein the semi-supervised or supervised machine learning algorithm comprises: a random forest, Bayesian model, a regression model, a neural network, a classification tree, a regression trees, discriminant analysis, a k-nearest neighbors method, a naive Bayes classifier, support vector machines (SVM), a generative model, a low-density separation method, a graph-based method, a heuristic approach, or a combination thereof.

31. The computer-implemented system of claim 29, wherein the machine learning algorithm is trained with a training set, each data point of the training set comprising a feature of the plurality of features, and a label, the label being positive, negative, or unlabeled.

32. The computer-implemented system of claim 31, wherein the training set comprises of no less than 50 training data points.

33. The computer-implemented system of claim 31, wherein the plurality of features comprises one or more categories of features selected from: RNA-based features, protein domain features, evolutionary features, mutability features, and splicing regulatory features.

34.-62. (canceled)

63. The computer-implemented system of claim 29, further comprising a semi-supervised or supervised machine learning classifier to differentiate between functional splicing regulatory elements and cryptic splicing regulatory elements of one or more of the alternative splicing events thereby predicting controllability of splicing, druggability and reversibility of aberrant splicing events.

64. The computer-implemented system of claim 63, wherein the predicting controllability of splicing, druggability and reversibility of aberrant splicing events is configured to be utilized for interpreting splicing events.

65. (canceled)

66. A computer-implemented method for quantifying a functional impact of alternative splicing events on protein structures, protein functions, RNA stability, RNA integrity, or biological pathways comprising:
(a) generating a plurality of features based on information stored in a database, wherein the information comprises metadata obtained from annotations of a plurality of types of alternative splicing based on public RNA-seq data or other biological data;
(b) obtaining one or more alternative splicing events;
(c) quantitatively estimating probabilities of the one or more alternative splicing events of damaging the protein structures, protein functions, RNA stability, RNA integrity, or biological pathways based on the plurality of features;
(d) applying a supervised or semi-supervised machine learning algorithm to predict the functional impact of the one or more alternative splicing events based on the estimated probabilities; and
(e) generating a list of prioritized and biologically relevant alternative splicing events based on prediction of the functional impact of the one or more alternative splicing events.

67. The computer-implemented method of claim 66, wherein the semi-supervised or supervised machine learning algorithm comprises: a random forest, Bayesian model, a regression model, a neural network, a classification tree, a regression tree, discriminant analysis, a k-nearest neighbors method, a naive Bayes classifier, support vector machines (SVM), a generative model, a low-density separation method, a graph-based method, a heuristic approach, or a combination thereof.

68. The computer-implemented method of claim 66, wherein the machine learning algorithm is trained with a training set, each data point of the training set comprising a feature of the plurality of features, and a label, the label being positive, negative, and unlabeled.

69. The computer-implemented method of claim 68, wherein the training set comprises of no less than 50 training data points.

70. The computer-implemented method of claim 66, wherein the plurality of features comprises one or more categories of features selected from: RNA-based features, protein domain features, evolutionary features, mutability features, and splicing regulatory features.

71. The computer-implemented method of claim 66, wherein the quantitatively estimating probabilities of the one or more alternative splicing events of damaging the protein structures, protein functions, RNA stability, RNA integrity, or biological pathways comprises quantitatively estimating damage caused by: removal of a functional protein domain by alternative splicing; nonsense-mediated decay (NMD) and translation frameshifting (FS) by alternative splicing; mutability of alternative splicing events; weighted closeness centrality of alternative splicing; or a combination thereof.

72. (canceled)

73. A method of identifying a disease condition comprising:
(a) identifying a splicing factor error;
(b) applying the computer-implemented method of claim 66 to analyze sequencing data with or without the splicing factor error wherein the sequencing data is from a database; and
(c) outputting a list of alternative splicing events promoted by the splicing factor error.

74.-81. (canceled)

81. The method of claim 73, wherein the disease condition is selected from a group consisting of cancer, leukemia, a disease of the central nervous system, muscular dystrophy, a hormonal disorder, chronic inflammation and abnormal inflammation.

82. The method of claim 73, wherein the disease condition is selected from a group consisting of familial dysautonomia (FD), Spinal muscular atrophy (SMA), Medium-chain acyl-CoA dehydrogenase (MCAD) deficiency, Hutchinson-Gilford progeria syndrome (HGPS), Myotonic dystophy Type 1 (DM1), Myotonic dystophy Type 2 (DM2), Autosomal dominant retinitis pigmentosa (RP), Duchenne muscular dystrophy (DMD), Microcephalic steodysplastic primordial dwarfism type 1 (MOPD1) or Taybi-Linder syndrome (TALS), Frontotemporal dementia with parkinsonism-17 (FTDP-17), Fukuyama congenital muscular dystrophy (FCMD), Amyotrophic lateral sclerosis (ALS), Hypercholesterolemia, and Cystic Fibrosis (CF).

83.-84. (canceled)

85. The method of claim 73, wherein the list of alternative splicing events comprises at least one gene of a group comprising: BRCA 1, BRCA2, EZH2, BIN1, BCL2L1, BCL2L11, CASP2, CCND1, CD44, ENAH, FAS, FGRF, HER2, HRAS, KLF6, MCL1, MKNK2, MSTR1, PKM, RAC1, RPS6KB1, VEGFA, IKBKAP, SMN2, MCAD, LMNA, DMPK, ZNF9, PRPF31, PRPF8, PRPF3, RP9, MAPT, TKTN, TPD-43, LDLR, CFTR, DMD, ATF2, and the gene encoding U4atac snRNA.

86. The method of claim 73, wherein a treatment regimen is recommended based on the list of AS events.

87. A computer-implemented method for identifying a disease-specific exon duo or exon trio comprising:
(a) receiving disease associated gene sequencing data from a source;
(b) differentiating known from novel annotations wherein the frequency, coverage, and source are extracted;
(c) assigning a reliability score to the disease-specific exon duo or exon trio based on the known annotations;
(d) sorting the annotations based on inclusion or skipping states;
(e) outputting a list of predicted exon duos and/or exon trios.

88.-99. (canceled)

100. A method of identifying an exon duo or exon trio associated with disease, the method comprising:
(a) applying the computer implemented method of claim 87 to database sequencing data on a mutation associated with disease;
(b) outputting a list of predicted exon duos and/or exon trios.

101. (canceled)
